(19) United States
(12) Patent Application Publication
(10) Pub. No.: US 2024/0075112 A1
(43) Pub. Date: Mar. 7, 2024

(54) SCAR TREATMENT COMPOSITION

(71) Applicant: Topix Pharmaceuticals, Inc., North Amityville, NY (US)

(72) Inventor: Steven M. Hernandez, Blue Point, NY (US)

(21) Appl. No.: 18/038,576

(22) PCT Filed: Nov. 24, 2021
(86) PCT No.: PCT/US2021/072591

§ 371 Date: May 24, 2023

Related U.S. Application Data
(60) Provisional application No. 63/118,045, filed on Nov. 25, 2020.

(51) Int. Cl. A61K 38/48; A61K 8/27; A61K 8/49; A61K 8/66; A61K 8/67; A61K 8/891; A61K 8/9789; A61K 9/06; A61K 31/355; A61K 31/4166; A61K 36/82; A61K 47/34; A61P 17/02; A61Q 17/04; A61Q 19/00; C12N 9/50

(52) U.S. Cl. CPC A61K 38/4873; A61K 8/27; A61K 8/4946; A61K 8/66; A61K 8/678; A61K 8/891; A61K 8/9789; A61K 9/06; A61K 31/355; A61K 31/4166; A61K 36/82; A61K 47/34; A61P 17/02; A61Q 17/04; A61Q 19/00; C12N 9/63; C12Y 304/22002; A61K 2800/522; A61K 2800/72

(57) ABSTRACT

Disclosed herein is a topical composition that treats and reduces the visual appearance of a scar through the combination of a hydrophobic occlusion forming agent with one or more proteolytic enzymes and an antioxidant system that includes antioxidant polyphenols (e.g., green tea polyphenols). Also disclosed herein are methods of administering the topical compositions described herein for treating and/or reducing the visual appearance of a scar by themselves or as part of a scar treatment regimen in combination with a variety of dermatological procedures. Processes of preparing the topical compositions disclosed herein are also described in certain embodiments.

### CROSS REFERENCE TO RELATED APPLICATION(S)

The present application claims priority to U.S. Provisional Application No. 63/118,045 filed on Nov. 25, 2020, the entire contents of which are incorporated in its entirety.

### FIELD OF THE INVENTION

The present invention relates to compositions for skin care applications and in particular for scar treatment.

### BACKGROUND OF THE INVENTION

Each year over 100 million patients acquire scars, mainly as a result of surgery and trauma (Bayat, 2003). Even scars from common skin conditions such as acne affect 40 to 50 million people in the U.S. alone (The Burden of Skin Diseases, 2004). Scars affect countless individuals from all geographies and demographics: many suffer psychological trauma, depression, stigmatization and overall decreased quality of life due to these disfiguring scars.

Most scars are comprised of fibrous tissue formed as a result of the human body's wound healing process following injury to the skin. Most every wound results in scarring to some degree. Scar severity can be affected by factors such as wound type, genetic predisposition, tissue tension or prolonged inflammation during wound healing. The human body's typical response to a wound is independent of wound causation; whether accidental or intentional, injuries caused by trauma, laser treatments, diseases or surgery are predisposed to heal following the same sequence of biological repair.

While most people try their best to avoid injury and surgery which might result in scars, sometimes trauma is inescapable. Strategies to minimize scars as they form include: maintaining a moist wound environment; removing tension from the healing wound; and refraining from the use of materials like latex or acrylic adhesives which might exacerbate the injury, as they are overly aggressive and can cause damage to delicate tissues, as well as reactions in pre-sensitized patients.

While prevention is preferable, there are situations in which the scar has already formed and treatment is clearly indicated. Scar treatment options vary and include, without limitations, medical devices, silicone gel sheeting, steroid injections, orthotic garments, elastic silicone dressing (Embrace®), topical scar preparations, pressure therapy, laser therapy, cryosurgery, dermabrasion, fractional radiofrequency skin rejuvenation, and scar revision surgery.

Despite the existing scar treatment options, there remains a need for a topical scar treatment composition that effectively improves the overall appearance and feel of a scar while having an aesthetically pleasing texture that would encourage patient compliance through a full duration of a recommended treatment regimen.

### SUMMARY

It is an object of certain embodiments of the disclosure to provide a topical composition, a method of preparing, and a method of using said topical composition for treating, preventing, or minimizing the appearance of a scar, reducing the redness or purple presentation of a scar, reducing scar volume, preventing the occurrence of a scar, improving flexibility of a scar, or a combination of two or more thereof.

It is an object of certain embodiments of the disclosure to provide a topical composition, a method of preparing, and a method of using said topical composition as part of a scar treatment regimen pre dermatological procedures or post dermatological procedures (such as dermatological procedures used for scar remediation, scar revisions, microneedling, dermaplaning, dermabrasion, microdermabrasion, and laser).

It is an object of certain embodiments of the disclosure to provide a topical composition for treating and/or improving the appearance of the skin in the scar area, treating and/or improving skin elasticity in the scar area, body firming or to treat and/or reduce the visible signs of photo-aged skin in the scar area, help treat and/or prevent sunburn in the scar area, decrease risk of skin cancer and early aging caused by the sun, and treat related skin disorders common to a scar area.

One or more of the above objects and others may be met in the instant disclosure which in certain embodiments is directed to a topical composition that includes an occlusion forming agent, one or more proteolytic enzymes, and an antioxidant system. The occlusion forming agent being hydrophobic (e.g., having less than about 25% water solubility). In some embodiments, the occlusion forming agent including one or more silicone based components. The antioxidant system includes polyphenol antioxidants (e.g., green tea polyphenols). The topical composition has less than 10 wt. % water, based on total weight of the topical composition.

In certain embodiments, the instant disclosure is directed to a topical composition that includes an occlusion forming agent, one or more proteolytic enzymes, and an antioxidant system. The occlusion forming agent being hydrophobic (e.g., having less than about 25% water solubility). In some embodiments, the occlusion forming agent including one or more silicone based components. The antioxidant system includes polyphenol antioxidants (e.g., green tea polyphenols). The topical composition has less than 8 wt. % urea, based on total weight of the topical composition.

In certain embodiments, the instant disclosure is directed to a topical composition that includes an occlusion forming agent, one or more proteolytic enzymes, and an antioxidant system, wherein the topical composition is a homogenous suspension. The occlusion forming agent being hydrophobic (e.g., having less than about 25% water solubility). In some embodiments, the occlusion forming agent including one or more silicone based components. The antioxidant system includes polyphenol antioxidants (e.g., green tea polyphenols).

The term “topical composition,” as used herein also refers to, in certain embodiment, a medical device that provides its therapeutic effect by physical means, instead of (or in addition to), pharmacological, immunological, or chemical means.

In certain embodiments, the instant disclosure is directed to a method of treating, preventing, or minimizing the appearance of a scar (e.g., the visual appearance of the scar, the color of the scar, the volume of the scar, improving the elasticity of the scar, reducing effects of radical-induced damage in a scar, or as part of a scar treatment regimen with certain dermatological procedures). These methods include administering, to the skin of the subject, in the scar area, any of the topical compositions described herein.

The term “administering the topical composition” as used herein refers to applying topically onto a skin of a subject, on the scar area, e.g., on the face, neck, hands, feet, elbows, knees, and the like. As used herein, the terms “application,” “apply,” and “applying” with respect to a disclosed topical formulation or method of using a disclosed topical formulation, refer to any manner of administering a topical formulation to the skin, for example, the skin of a person, such as the skin of a patient, which, in medical or cosmetology practice, delivers the formulation to the subject's skin surface. Smearing, rubbing, spreading, spraying a disclosed topical formulation, with or without the aid of suitable devices, on a subject's skin are all included within the scope of the term “application,” as used herein. The term “topical” or “topically” with respect to administration or application of a disclosed skincare formulation refers to epicutaneous administration or application, onto skin. The application can be manually (e.g., directly with the hands) or manipulated with an applicator, cloth, device, roll-on, wipes, unit dose sponge applicators, liquid applied with swabs or cotton balls, impregnated gauze or other substrates, coated silicone sheets or other sheet goods, coated bandages or externally fixed devices, towelettes, individually packages pledgettes or pads, transdermal delivery system, etc. Administration can be self-administration or administration by a medical professional or caregiver.

In certain embodiments, the instant disclosure is directed to a process of preparing any of the topical compositions described herein by forming a first mixture including the occlusion forming agent, which may include a hydrophobic occlusion agent having less than about 25% solubility in water (e.g., one or more silicone based components). The process may further include forming a second mixture including one or more proteolytic enzymes. The process may further include combining the first mixture and the second mixture to form a third mixture. The process may further include adding an antioxidant system, which may include antioxidant polyphenols (e.g., green tea polyphenols), to the third mixture.

### BRIEF DESCRIPTION OF THE DRAWINGS

The following drawings are herein presented in the application:

FIG. 1A is a photo of a fresh wound.

FIG. 1B is a photo of the fresh wound of FIG. 1A after treatment with a scar treatment ointment according to an embodiment of the present disclosure.

FIG. 2A is a photo of a side of the head after a MOHS surgery.

FIG. 2B is a photo of the side of the head in FIG. 2A 2 weeks after treatment using a hydrophilic scar treatment composition.

FIG. 2C is a photo of the side of the head in FIG. 2A 6 weeks after treatment using the hydrophilic scar treatment composition and the scar treatment ointment according to an embodiment of the present disclosure.

FIG. 2D is a photo of the side of the head in FIG. 2A 12 weeks after treatment using the hydrophilic scar treatment composition and the scar treatment ointment according to an embodiment of the present disclosure.

FIG. 3A is a photo of a hand immediately after a MOHS surgery.

FIG. 3B is a photo of the hand in FIG. 3A 2 weeks after treatment using a hydrophilic scar treatment composition.

FIG. 3C is a photo of the hand in FIG. 3C 12 weeks after treatment using the hydrophilic scar treatment composition and the scar treatment ointment according to an embodiment of the present disclosure.

FIG. 4A is a photo of a hand immediately after a MOHS surgery.

FIG. 4B is a photo of the hand in FIG. 4A 12 weeks after treatment using the hydrophilic scar treatment composition and the scar treatment ointment according to an embodiment of the present disclosure.

FIG. 5A is a photo of a forehead immediately after a MOHS surgery.

FIG. 5B is a photo of the forehead in FIG. 5A 2 weeks after treatment using a hydrophilic scar treatment composition.

FIG. 5C is a photo of the forehead in FIG. 5A 8 weeks after treatment using a hydrophilic scar treatment composition and the scar treatment ointment according to an embodiment of the present disclosure.

FIG. 5D is a photo of the forehead in FIG. 5A 12 weeks after treatment using a hydrophilic scar treatment composition and the scar treatment ointment according to an embodiment of the present disclosure.

### DETAILED DESCRIPTION

The following definitions are used, unless otherwise described.

The term “alkyl” as used herein refers to straight and branched hydrocarbon groups. Reference to an individual radical such as propyl embraces only the straight chain radical, a branched chain isomer such as isopropyl being specifically referred to.

The term “halo” or “halogen” as used herein refers to fluoro, chloro, bromo and iodo.

The term “carbocycle” or “carbocyclyl” refers to a single saturated (i.e., cycloalkyl) or a single partially unsaturated (e.g., cycloalkenyl, cycloalkadienyl, etc.) ring having 3 to 7 carbon atoms (i.e. (C₃-C₇)carbocycle). The term “carbocycle” or “carbocyclyl” also includes multiple condensed ring systems (e.g. ring systems comprising 2, 3 or 4 carbocyclic rings). Accordingly, carbocycle includes multicyclic carbocyles having 7 to 12 carbon atoms as a bicycle, and up to about 20 carbon atoms as a polycycle. Multicyclic carbocyles can be connected to each other via a single carbon atom to form a spiro connection (e.g. spiropentane, spiro[4,5]decane, spiro[4.5]decane, etc.), via two adjacent carbon atoms to form a fused connection such as a bicyclo[4,5], [5,5], [5,6] or [6,6] system, or 9 or 10 ring atoms arranged as a bicyclo[5,6] or [6,6] system (e.g. decahydronaphthalene, norsabinane, norcarane) or via two non-adjacent carbon atoms to form a bridged connection (e.g. norbornane, bicyclo[2.2.2]octane, etc.). The “carbocycle” or “carbocyclyl” may also be optionally substituted with one or more (e.g. 1, 2 or 3) oxo groups. Non-limiting examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl and cycloheptyl.

The term “aryl” as used herein refers to a single aromatic ring or a multiple condensed ring system. For example, an aryl group can have 6 to 20 carbon atoms, 6 to 14 carbon atoms, or 6 to 12 carbon atoms. Aryl includes a phenyl radical. Aryl also includes multiple condensed ring systems (e.g. ring systems comprising 2, 3 or 4 rings) having about 9 to 20 carbon atoms in which at least one ring is aromatic. Such multiple condensed ring systems may be optionally substituted with one or more (e.g. 1, 2 or 3) oxo groups on any carbocycle portion of the multiple condensed ring system. It is to be understood that the point of attachment of a multiple condensed ring system, as defined above, can be at any position of the ring system including an aryl or a carbocycle portion of the ring. Typical aryl groups include, but are not limited to, phenyl, indenyl, naphthyl, 1,2,3,4-tetrahydronaphthyl, anthracenyl, and the like.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

As used herein, “free or substantially free,” refers to a subject composition (e.g., topical composition) that comprises less than about 1 wt. %, less than about 0.5 wt. %, less than about 0.25 wt. %, less than about 0.1 wt. %, less than about 0.05 wt. %, less than about 0.01 wt. %, or 0 wt. % of said component.

As used herein, “hydrophobic,” refers to a component having a water solubility of less than about 50%, less than about 40%, less than about 30%, less than about 25%, less than about 20%, less than about 15%, less than about 10%, less than about 5%, less than about 4%, less than about 3%, less than about 2%, less than about 1%, or less than about 0.5%. For example, less than 50% water solubility would mean that less than 50 g of said component would dissolve in 100 ml water.

### Topical Composition

According to various embodiments, the present disclosure is related to a topical composition for the treatment of a scar. The topical composition includes an occlusion forming agent, e.g., a hydrophobic occlusion forming agent, that includes, e.g., one or more silicone based components, one or more proteolytic enzymes, and an antioxidant system that includes polyphenol antioxidants (e.g., green tea polyphenols).

The occlusion forming agent in the topical compositions described herein helps to mechanically soften the scar, diminish the scar in volume, and form a seal over the scar. The occlusion that is formed over the scar (due to the presence of the occlusion forming agent) helps the skin to sweat under the occlusion, which opens the skin's induction channels and enables quicker transdermal uptake (i.e., increase transdermal flux rate of the proteolytic enzymes under the occlusion). In this manner, the one or more proteolytic enzymes, that are present in the topical compositions described herein, are forced into the skin in order to digest the skin in the scar area and/or cause the skin in the scar area to dissolve or turn over quicker. The uptake of proteolytic enzyme(s) into the skin could encourage further remodeling of the scar to remove excess tissue, and align the tissue in a more organized and dense manner, which would improve the scar's appearance (e.g., in terms of volume, shape, and color).

Proteolytic enzyme(s) may be quite aggressive, which is beneficial for breaking the very dense scar tissue on the one hand, but can cause irritation and reduce patient compliance of continuous administration of the composition on the other hand. The antioxidant system with the antioxidant polyphenols (e.g., green tea polyphenols), that are present in the topical compositions described herein, soothe the skin under the occlusion by preventing and/or mitigating irritation, stinging, burning, and/or itching sensation that may be associated with the proteolytic enzymes. The soothing effect of the antioxidant system that is incorporated into the topical compositions described herein increases patient compliance, providing for continuous administration of the topical composition over a duration needed to treat scar related skin disorders and/or to achieve improvement in the scar's visual appearance.

Various components of the topical compositions described herein will be discussed below in detail. It should be noted that, in certain embodiments, various components may be substituted by other similar or equivalent components, as will be appreciated by one skilled in the art. Similarly, the components may be present at various concentrations and at various ratios in relation to each other, to achieve any of the objectives described herein.

In certain embodiments, suitable occlusion forming agents that may be utilized in the topical compositions described herein are hydrophobic. For instance, in some embodiments, the occlusion forming agent(s) have a water solubility of less than about 50%, less than about 40%, less than about 30%, less than about 25%, less than about 20%, less than about 15%, less than about 10%, less than about 5%, less than about 4%, less than about 3%, less than about 2%, less than about 1%, or less than about 0.5%. In certain embodiments, the occlusion forming agent(s) have a water solubility of less than about 25%, less than about 20%, less than about 15%, less than about 10%, less than about 5%, less than about 4%, less than about 3%, less than about 2%, less than about 1%, or less than about 0.5%. In one embodiment, the occlusion forming agent(s) have a water solubility of less than about 25%. In one embodiment, the occlusion forming agent(s) have a water solubility of less than about 15%. In one embodiment, the occlusion forming agent(s) have a water solubility of less than about 5%. In one embodiment, the occlusion forming agent(s) have a water solubility of less than about 1%.

In various embodiments, various hydrophobic occlusion forming agent(s) may be used, such as, without limitations, one or more silicone based components, lipids, Vaseline, petrochemical grease, or an oily constituent (e.g., certain esters, straight hydrocarbons, squalene), or a combination thereof. Any of these hydrophobic occlusion agents may be combined, if needed, with a gelling agent or a thickener, such as a polymer or a waxy component to attain a target consistency for the topical composition.

In one embodiment, suitable occlusion forming agents that may be utilized in the topical compositions described herein include, without limitations, one or more silicone based components, such as, a cosmetic wax (e.g., C30-45 alkyl methicone), a silicone resin, phenyl trimethicone, or a combination thereof.

Although silicone resin is an effective occlusion forming agent for scar treatment, when it is present in a composition at high concentrations, it may create a gel like consistency that could stain clothes, be sticky, and have a generally unpleasant texture that could potentially adversely affect patient's compliance with respect to continuous administration.

In certain embodiments, a cosmetic wax, such as C30-45 alkyl methicone, is added to the topical composition due to its texture, which gives the composition a pleasant feel that is less aggressive, sticky, or tacky, as compared to topical compositions that include predominantly silicone.

In certain embodiments, the topical composition includes a combination of a cosmetic wax, such as C30-45 alkyl methicone, and a silicone resin, as the occlusion forming agents. In certain embodiments, the topical composition includes a cosmetic wax, such as C30-45 alkyl methicone, a silicone resin, and optionally a phenyl trimethicone, at an effective amount to form a topical composition capable of forming an occlusion upon application to a scar, while still having an aesthetically pleasing texture (e.g., washable, less sticky, less tacky, and the like) to encourage continuous application for a duration needed to see improvement in a scar.

The occlusion forming agent in any of the topical compositions described herein may be present in a concentration (individual or cumulative if more than one occlusion forming agent is included) of from any of about 1 wt. %, about 3 wt. %, about 5 wt. %, about 8 wt. %, about 10 wt. %, about 15 wt. %, about 20%, about 25 wt. %, about 30 wt. %, or about 35 wt. % to any of about 40 wt. %, about 45 wt. %, about 50 wt. %, about 55 wt. %, about 60 wt. %, about 65 wt. %, about 70 wt. %, about 75 wt. %, about 80 wt. %, about 85 wt. %, about 90 wt. %, about 95 wt. %, about 99 wt. %, about 99.5 wt. %, about 99.7 wt. %, or about 99.9 wt. %, based on total weight of the topical composition.

In certain embodiments, the occlusion forming agent includes a cosmetic wax, such as C30-45 alkyl methicone, that is present in any of the topical compositions described herein in an amount ranging from about 1 wt. % to about 30 wt. %, from about 2 wt. % to about 15 wt. %, or from about 3 wt. % to about 5 wt. %, based on total weight of the topical composition.

In certain embodiments, the occlusion forming agent includes a silicone resin that is present in any of the topical compositions described herein in an amount ranging from about 2.5 wt. % to about 30 wt. %, from about 5 wt. % to about 20 wt. %, or from about 7.5 wt. % to about 15 wt. %, based on total weight of the topical composition.

In certain embodiments, the occlusion forming agent includes phenyl trimethicone that is present in any of the topical compositions described herein in an amount ranging from above 0 wt. % to about 30 wt. %, from about 2 wt. % to about 20 wt. %, or from about 5 wt. % to about 10 wt. %, based on total weight of the topical composition. While the phenyl trimethicone is not essential to the topical compositions described herein, it does improve the texture of the topical composition and enhances patient compliance with continuous application of the topical composition.

In certain embodiments, the weight to weight ratio of the silicone resin to the cosmetic wax may range from any of about 10:1, about 8:1, about 5:1, about 3:1, about 2:1, or about 1:1 to any of about 1:2, about 1:3, about 1:5, about 1:8, or about 1:10. The weight to weight ratio of the silicone resin to the cosmetic wax may be such that would achieve an aesthetically pleasing texture to the composition while still remaining efficacious in scar treatment.

In certain embodiments, the weight to weight ratio of the silicone resin to the phenyl trimethicone may range from any of about 10:1, about 8:1, about 5:1, about 3:1, about 2:1, or about 1:1 to any of about 1:2, about 1:3, about 1:5, about 1:8, or about 1:10.

In certain embodiments, the weight to weight ratio of the silicone resin to the cumulative weight of all occlusion forming agents in the topical composition may range from any of about 1:1, about 1:2, or about 1:3 to any of about 1:4, about 1:5, or about 1:6.

In certain embodiments, suitable proteolytic enzymes that may be utilized in the topical compositions described herein include, without limitations, bromelain, papain, or a combination thereof. In one embodiment, the proteolytic enzyme in the topical composition is bromelain. In one embodiment, the proteolytic enzyme in the topical composition is papain. In one embodiment, the proteolytic enzyme in the topical composition is a combination of bromelain and papain.

In certain embodiments, the one or more proteolytic enzymes in the topical composition may be present in an effective amount for scavenging over production of scar forming proteins and tissues.

In certain embodiments, the one or more proteolytic enzyme(s) is(are) present in any of the topical compositions described herein in an amount (individual or cumulative) ranging from above 0 wt. % to about 1 wt. %, from about 0.001 wt. % to about 0.5 wt. %, or from about 0.005 wt. % to about 0.3 wt. %, based on total weight of the topical composition.

When the combination of papain and bromelain is present in the topical compositions described herein, the weight to weight ratio of the papain to bromelain may range from any of about 10:1, about 8:1, about 5:1, about 3:1, about 2:1, or about 1:1 to any of about 1:2, about 1:3, about 1:5, about 1:8, or about 1:10.

As mentioned above, uptake of proteolytic enzymes by the skin could create irritation, stinging, burning, and/or itching sensation which could be quite aggressive and discourage subjects for continuously applying the composition. It was discovered that incorporation of an antioxidant system that includes polyphenol antioxidants, such as green tea polyphenols, soothes the skin (e.g., reduces redness, swelling, and/or irritation), improves patient's compliance, and the overall efficacy of the topical compositions described herein in scar treatment. In certain embodiments, polyphenols derived from sources other than green tea can also be suitably utilized in the topical compositions described herein (e.g., in addition or as an alternative to the green tea polyphenols).

Certain plants, such as Camellia sinensis (green tea), that are composed of a high content of polyphenols which are bioflavonoids and have antioxidant properties may be included in any of the topical compositions described herein. The antioxidant system in the topical compositions described herein may be present in a concentration of from any of above 0%, about 0.01 wt. %, about 0.05 wt. %, about 0.1 wt. %, about 0.15 wt. %, about 0.2 wt. %, about 0.25 wt. %, about 0.3 wt. %, about 0.35 wt. %, or about 0.4 wt. % to any of about 0.45 wt. %, about 0.5 wt. %, about 0.55 wt. %, about 0.6 wt. %, about 0.65 wt. %, about 0.7 wt. %, about 0.75 wt. %, about 0.8 wt. %, about wt. %, about 0.9 wt. %, about 0.95 wt. %, about 1 wt. %, about 1.5 wt. %, about 2 wt. %, about 3 wt. %, about 4 wt. %, about 5 wt. %, about 6 wt. %, about 7 wt. %, about 8 wt. %, about 9 wt. %, or about 10 wt. %, based on total weight of the topical composition.

In certain embodiments, the topical composition includes an effective amount of polyphenol antioxidants, to assist in soothing the skin in the scar area. In certain embodiments, the topical composition includes an effective amount of green tea polyphenol isolates, derived from green tea with potent antioxidant properties, to assist in soothing the skin in the scar area. Suitable green tea polyphenols include, but are not limited to, catechins, such as epigallocatechin gallate (EGCG), epigallocatechin (EGC), epicatechin gallate (ECG), and epicatechin (EC), cis and trans isomers thereof, salts thereof, equivalent derivatives thereof, and combinations thereof.

In certain embodiments, the topical composition includes an antioxidant system which includes any of the green tea polyphenols described herein in combination with at least one additional antioxidants.

In one embodiment, the additional antioxidants in the antioxidant system may be selected from cinnamic acid, ferulic acid, caffeic acid, p-coumaric acid, sinapinic acid, cis and trans isomers thereof, salts thereof, equivalent derivatives thereof, or combinations thereof. In another embodiment, the additional antioxidants in the antioxidant system (and in the topical composition generally) may be free or substantially free of cinnamic acid, ferulic acid, caffeic acid, p-coumaric acid, sinapinic acid, cis and trans isomers thereof, salts thereof, equivalent derivatives thereof, or combinations thereof.

In certain embodiments, the additional antioxidants in the antioxidant system may be selected from resveratrol, gallic acid, delphinidin, luteolin, quercetin, cyanidin, taxifolin, kaempferol, malvidin, hesperidin, pelargonidin, apigenin, naringenin, chrysin, ergothioneine, glutathione, emblica, vitamin E (tocopherol), allantoin, cis and trans isomers thereof, salts thereof, equivalent derivatives thereof, or combinations thereof.

Any of the above antioxidants may also, in certain embodiments, be used in any of the topical compositions described herein as an alternative to green tea polyphenols.

In certain embodiments, the additional antioxidants in the antioxidant system may be selected from tocopherol, allantoin, or combinations thereof. The antioxidant system may include a combination of antioxidant(s) that will be taken up by the fatty tissue and antioxidant(s) that will be taken up by the hydrophilic interstitial fluids of the skin.

Each of the antioxidants in the antioxidant system may be present, individually or cumulatively, in a concentration of from any of above 0 wt. %, about 0.01 wt. %, about 0.05 wt. %, about 0.1 wt. %, about 0.15 wt. %, about 0.2 wt. %, about 0.25 wt. %, about 0.3 wt. %, about wt. %, or about 0.4 wt. % to any of about 0.45 wt. %, about 0.5 wt. %, about 0.55 wt. %, about wt. %, about 0.65 wt. %, about 0.7 wt. %, about 0.75 wt. %, about 0.8 wt. %, about 0.85 wt. %, about 0.9 wt. %, about 0.95 wt. %, about 1 wt. %, about 1.5 wt. %, about 2 wt. %, about 3 wt. %, about 4 wt. %, about 5 wt. %, about 6 wt. %, about 7 wt. %, about 8 wt. %, about 9 wt. %, or about wt. %, based on total weight of the topical composition.

In certain embodiments, the cosmetically acceptable excipient includes additional antioxidants such as a form of Vitamin E. Suitable forms of Vitamin E that may be included in the topical composition can be selected from alpha, beta, delta, and gamma tocopherols, and alpha, beta, delta and gamma tocotrienols, and combinations thereof. In certain embodiments, the antioxidant system includes Vitamin E that is present in the topical composition in an amount ranging from above 0 wt. % to about 0.5 wt. %, from above 0 wt. % to about 0.1 wt. %, or from above 0 wt. % to about 0.02 wt. %, based on total weight of the topical composition.

In certain embodiments, the antioxidant system includes allantoin that is present in the topical composition in an amount ranging from above 0 wt. % to about 0.5 wt. %, from above 0 wt. % to about 0.1 wt. %, or from above 0 wt. % to about 0.02 wt. %, based on total weight of the topical composition.

In certain embodiments, the antioxidant system includes green tea polyphenols in an amount ranging from above 0 wt. % to about 0.5 wt. %, from above 0 wt. % to about 0.1 wt. %, or from above 0 wt. % to about 0.05 wt. %, based on total weight of the topical composition.

In certain embodiments, the antioxidant system includes, comprises, consists, or consists essentially of a combination of any of the green tea polyphenols described herein and at least one of tocopherol and allantoin in an (individual or cumulative) concentration of from any of above 0 wt. %, about 0.01 wt. %, about 0.05 wt. %, about 0.1 wt. %, about 0.15 wt. %, about 0.2 wt. %, about 0.25 wt. %, about 0.3 wt. %, about 0.35 wt. %, or about 0.4 wt. % to any of about 0.45 wt. %, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, about 1 wt. %, about 1.5 wt. %, about 2 wt. %, about 3 wt. %, about 4 wt. %, about 5 wt. %, about 6 wt. %, about 7 wt. %, about 8 wt. %, about 9 wt. %, or about 10 wt. %, based on total weight of the topical composition.

In certain embodiments, the weight to weight ratio of the green tea polyphenols to the one or more additional antioxidants (individually or cumulatively) ranges from about 10:1 to about 1:10, about 8:1 to about 1:8, about 5:1 to about 1:5, about 3:1 to about 1:3, about 2:1 to about 1:2, or about 1:1.

In certain embodiments, any of the topical compositions described herein are hydrophobic and include less than about 10 wt. % water, less than about 8 wt. % water, less than about 5 wt. % water, less than about 3 wt. % water, less than about 2 wt. %, less than about 1 wt. % water, or is free of water, based on total weight of the topical composition. In certain embodiments, the upper limit of the water is reflective of the maximum carrying capacity of the topical composition beyond which the topical composition may exhibit different properties than those described herein. In certain embodiments, the upper limit of the water is reflective of the water content in the topical composition due to moisture that may be present in the raw materials used to form the topical composition.

In certain embodiments, any of the topical compositions described herein include less than about 8 wt. % urea or urea derivatives, less than about 5 wt. % urea or urea derivatives, less than about 3 wt. % urea or urea derivatives, less than about 1 wt. % urea or urea derivatives, or is free of urea or urea derivatives, based on total weight of the topical composition. Urea derivatives contemplated by the present disclosure include, but are not limited to, derivatives of urea that have similar function or act in the same way as urea when used in skin care treatments, such as dimethylurea, dimethylolurea, hydroxyethyl urea, hydroxyethoxyethyl urea, among others.

In certain embodiments, topical compositions contemplated by the instant disclosure are hypoallergenic. As such, these topical composition may be utilized on sensitive skin while remaining substantially unlikely to cause an allergic reaction.

In certain embodiments, topical compositions contemplated by the instant disclosure are non-comedogenic. As such, these topical composition do not cause blocked pores when applied to the skin of a subject. In certain embodiments, topical compositions described herein are substantially free of ingredients that may cause clogged pores, such as, without limitations, petrolatum, jojoba oil, olive oil, avocado oil, isopropyl myristate, isopropyl palmitate, ethylhexyl palmitate, propylene glycol-2 (PPG-2) myristyl propionate, acetylated lanolin, tar, artificial colors, and the like.

In certain embodiments, the topical compositions described herein have an initial viscosity at 25° C., measured with a viscometer, model LVT, with a TF heliopath spindle at 60 RPM and 1 minute reading, of about 500,000 cPs to about 1,100,000 cPs, about 520,000 cPs to about 1,050,000 cPs, or about 540,000 cPs to about 1,014,000 cPs.

In certain embodiments, the topical compositions described herein have a specific gravity of about 1.00 to about 1.04, about 1.01 to about 1.035, or about 1.02 to about 1.03.

In certain embodiments, the topical compositions described herein include at least one additional cosmetically acceptable excipient. Exemplary cosmetically acceptable excipients, include, without limitations, epidermal penetration enhancer, solvent, mild surfactants, oil bodies, emulsifiers, pearlescent waxes, consistency regulators, thickeners (e.g., glyceryl behenate), rheology modifiers, suspending agents, chelating agents, preservatives, super fatting agents, stabilizers or carriers (e.g., isononyl isononanoate, a silicone organic elastomer blend of isodecyl neopentanoate and dimethicone/bis-isobutyl PPG-20 cross polymer, HDI/trimethylol hexyllactone crosspolymer and silica), polymers, additional silicone or siloxane compounds, fats, waxes, lecithins, phospholipids, UV photoprotective factors, biogenic active ingredients, additional antioxidants, deodorants, antiperspirants, antidandruff agents, film formers, swelling agents, insect repellents, self-tanning agents, tyrosinase inhibitors, hydrotropes, solubilizers, perfume oils, dyes, zinc oxide, fatty alcohols, esters of fatty acids, adjuvants, Natural or Synthetic Triglycerides Including Glyceryl Esters and Derivatives, hydrocarbon oils, super-fatting agents, hydrotropic agents, bacteria-inhibiting agents, colorants, UV screening agents, agents that absorb UV light and provide photo protection to the skin, preservatives (e.g., phenoxyethanol), or combinations thereof. In certain embodiments, the topical composition includes a cosmetically acceptable excipient selected from the group of thickeners, preservatives, stabilizers or carriers, or a combination thereof.

In certain embodiments, the topical compositions described herein include a thickener that is glyceryl behenate at an amount of about 1 wt. % to about 15 wt. %, about 2.5 wt. % to about 10 wt. %, or about 4 wt. % to about 7.5 wt. %, based on total weight of the topical composition.

In certain embodiments, the cosmetically acceptable excipient in the topical composition includes a preservative. Suitable preservative agents include, for example, phenoxyethanol, a solution of paraben, pentanediol and sorbic acid, as well as silver complexes which are known under the commercial reference Surfacine® and other classes of substances set out in annex 6, parts A and B of the cosmetic regulations, i.e. a suitable preservative. In certain embodiments, the topical compositions described herein include a preservative that is phenoxyethanol at an amount of up to about 2 wt. %, up to about 1.5 wt. %, or up to about 0.5 wt. %, based on total weight of the topical composition. In certain embodiments, the topical compositions described herein are substantially free or free or preservatives.

In certain embodiments, the topical compositions described herein include one or more stabilizer or carrier, such as, isononyl isononanoate, a silicone organic elastomer blend of isodecyl neopentanoate and dimethicone/bis-isobutyl PPG-20 cross polymer, or a combination thereof. In certain embodiments, the topical compositions described herein include isononyl isononanoate at an amount of about 5 wt. % to about 40 wt. %, about 10 wt. % to about 30 wt. %, or about 15 wt. % to about 20 wt. %, based on total weight of the topical composition. In certain embodiments, the topical compositions described herein include a silicone organic elastomer blend of isodecyl neopentanoate and dimethicone/bis-isobutyl PPG-20 cross polymer at an amount of about 5 wt. % to about 40 wt. %, about 10 wt. % to about 35 wt. %, or about 20 wt. % to about 30 wt. %, based on total weight of the topical composition.

Stabilizers or carriers that are incorporated in the topical compositions described herein enhance the aesthetic texture of the composition, rendering the topical compositions silky, light, dry, palatable, not greasy or tacky, and readily washable. Suitable stabilizers or carriers are also those that can effectively suspend the occlusion forming agents, such as the one or more silicone based components.

For instance, the silicone organic elastomer blend of isodecyl neopentanoate and dimethicone/bis-isobutyl PPG-20 cross polymer has the ability to combine together the various constituents of the topical compositions described herein to generate a homogenous suspension in a form of an ointment suitable for topical administration for scar treatment. This particular elastomer blend assists in incorporation of esters (including esters that may be present in certain sunscreens if those are added into the composition) so that they do not separate out from other constituents of the topical composition (such as the silicone based components). The elastomer backbone in this elastomer blend has been modified to include hydrophilic and hydrophobic moieties allowing it to complex with esters, silicones, and inorganic components, to arrive at a homogenous suspension.

In certain embodiments, the topical compositions described herein include at least one proteolytic enzyme (e.g., papain, bromelain, or a combination thereof), at least one antioxidant (e.g., Allantoin, Tocopherol, Polyphenols such as Camellia Sinensis (Green Tea) Polyphenols, or a combination thereof), and one or more of: C12-15 Alkyl Benzoate, C30-45 Alkyl Methicone, C30-45 Olefin, Dimethicone, Glyceryl Behenate, HDI/trimethylol hexyllactone crosspolymer and silica, silicone organic elastomer blend of isodecyl neopentanoate and dimethicone/bis-isobutyl PPG-20, isononyl isononanoate, isostearic acid, phenoxyethanol, phenyl trimethicone, polyhydroxystearic acid, silica, and trimethylsiloxysilicate.

The topical compositions described herein may be formulated in any dermatological acceptable vehicle such as an ointment, serum, emulsion, cream, foam, spray, ointment, gel, lotion, or as a pad or roll-on applied formulation, which may contain ingredients to improve, modify, or stabilize the composition physically or cosmetically.

The topical compositions according to the disclosure may also contain one or one more additional cosmetically acceptable excipients as described below.

### Solvents

In certain embodiments, cosmetically acceptable excipients that may be used in the topical compositions described herein include, without limitations, polysorbate 20, water, alkanediols (e.g., ethylene glycol, propylene glycol, butylene glycol), ethoxylated or propoxylated diglycol, ethanol, propanol, isopropanol, glycerin, methoxyisopropanol, PPG-2 methyl ether, PPG-3 methyl ether, propylene glycol butyl ether, PPG-2 butyl ether, phenoxyisopropanol, butoxyethanol, butoxydiglycol, methoxydiglycol, phenoxyethanol, PPG-3 butyl ether, PPG-2 propyl ether, propylene glycol propyl ether, or dipropylene glycol dimethyl ether, or mixtures and combinations or individual ethoxylates, propoxylates and glyceryl esters thereof.

### Gums

In certain embodiments, the cosmetically acceptable excipient includes natural gums (e.g., a natural plant gum). Suitable natural gums include, without limitations, guar gum, carob gum, konjac gum, xanthan gum, sclerotium gum, acacia gum, cellulose gum (modified or not), or a combination thereof.

### Chelating Agents

In certain embodiments, the cosmetically acceptable excipient includes a chelating agent. Suitable chelating agents include, without limitations, disodium ethylenediaminetetraacetic acid (EDTA), diethyl enetriaminepentaacetic acid (DTPA), N-(hydroxyethyl)-ethylenediaminetriacetic acid (HEDTA), and nitrilotriacetic acid (NTA).

### Perfume Oils

In certain embodiments, the cosmetically acceptable excipient includes a perfume oil. Suitable perfume oils include mixtures of natural and synthetic fragrances. Natural fragrances are extracts from flowers (lily, lavender, rose, jasmine, neroli, ylang-ylang), stems and leaves (geranium, patchouli, petitgrain), fruits (aniseed, coriander, cumin, juniper), fruit peels (bergamot, lemon, orange), roots (mace, angelica, celery, cardamom, costus, iris, calmus), woods (pinewood, sandalwood, guaiac wood, cedarwood, rosewood), herbs and grasses (tarragon, lemongrass, sage, thyme), needles and branches (spruce, fir, pine, dwarf-pine), resins and balsams (galbanum, elemi, benzoin, myrrh, olibanum, opoponax). Typical synthetic fragrance compounds are products of the ester, ether, aldehyde, ketone, alcohol and hydrocarbon type. Fragrance compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl-methylphenyl glycinate, allyl cyclohexylpropionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamen aldehyde, hydroxycitronellal, lilial and bourgeonal, and the ketones include, for example, the ionones, α-isomethylionone and methyl cedryl ketone, the alcohols include anethole, citronellol, eugenol, isoeugenol, geraniol, linalool, phenylethyl alcohol and terpineol, and the hydrocarbons include mainly the terpenes and balsams.

Essential oils of relatively low volatility, which are mostly used as aroma components, are also suitable as perfume oils, e.g. sage oil, chamomile oil, oil of cloves, melissa oil, mint oil, cinnamon leaf oil, linden blossom oil, juniper berry oil, vetiver oil, olibanum oil, galbanum oil, labolanum oil and lavandin oil. Other suitable oils include bergamot oil, dihydromyrcenol, lilial, lyral, citronellol, phenylethyl alcohol, α-hexylcinnamaldehyde, geraniol, benzylacetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix coeur, iso-E-super, Fixolide NP, evernyl, iraldein gamma, phenylacetic acid, geranyl acetate, benzyl acetate, rose oxide, romilat, irotyl and floramat alone or in mixtures.

In certain embodiments, the cosmetically acceptable excipient includes a perfume oil that is an essential oil selected from the group of a lavender oil, a bergamot oil, a Eucalyptus oil, a chamomile oil, a Melaleuca oil, or a combination thereof.

### Fatty Alcohols

Guerbet alcohols based on fatty alcohols having from 6 to 18, preferably from 8 to 10 carbon atoms including cetyl alcohol, stearyl alcohol, cetearyl alcohol, oleyl alcohol, octyldodecanol, benzoate of C12-C15 alcohols, acetylated lanolin alcohol, etc.

### Esters of Fatty Acids

Esters of linear C₆-C₂₄ fatty acids with linear C₃-C₂₄ alcohols, esters of branched C₆-C₁₃carboxyl acids with linear C₆-C₂₄ fatty alcohols, esters of linear C₆-C₂₄ fatty acids with branched alcohols, especially 2-ethylhexanol, esters of hydroxycarboxylic acids with linear or branched C₆-C₂₂ fatty alcohols, especially dioctyl malates, esters of linear and/or branched fatty acids with polyhydric alcohols (for example propylene glycol, dimer diol or trimer triol) and/or Guerbet alcohols, for example caproic acid, caprylic acid, 2-ethylhexanoic acid, capric acid, lauric acid, isotridecanoic acid, myristic acid, palmitic acid, palmitoleic acid, stearic acid, isostearic acid, polyhydroxystearic acid, oleic acid, elaidic acid, petroselinic acid, linoleic acid, linolenic acid, elaeostearic acid, arachidic acid, gadoleic acid, behenic acid and erucic acid and technical-grade mixtures thereof (obtained, for example, in the pressure removal of natural fats and oils, in the reduction of aldehydes from Roelen's oxosynthesis or in the dimerization of unsaturated fatty acids) with alcohols, for example, isopropyl alcohol, caproic alcohol, capryl alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linoyl alcohol, linolenyl alcohol, elaeostearyl alcohol, arachidyl alcohol, gadoleyl alcohol, behenyl alcohol, erucyl alcohol and brassidyl alcohol and technical-grade mixtures thereof (obtained, for example, in the high-pressure hydrogenation of technical-grade methyl esters based on fats and oils or aldehydes from Roelen's oxosynthesis and as monomer fractions in the dimerization of unsaturated fatty alcohols).

Examples of such ester oils are isopropyl myristate, isopropyl palmitate, isopropyl stearate, isopropyl isostearate, isopropyl oleate, n-butyl stearate, n-hexyl laurate, n-decyl oleate, isooctyl stearate, iso-nonylstearate, isononyl isononanoate, 2-ethylhexylpalmitate, 2-hexyllaurate, 2-hexyldecylstearate, 2-octyldodecylpalmitate, oleyloleate, oleylerucate, erucyloleate, erucylerucate, cetearyl octanoate, cetyl palmitate, cetyl stearate, cetyl oleate, cetyl behenate, cetyl acetate, myristyl myristate, myristyl behenate, myristyl oleate, myristyl stearate, myristyl palmitate, myristyl lactate, propylene glycol dicaprylate/caprate, stearyl heptanoate, diisostearyl malate, octyl hydroxystearate, etc.

### Other Adjuvants

Diethylhexyl 2,6-naphthalate, di-n-butyl adipate, di(2-ethylhexyl)-adipate, di(2-ethyl hexyl)-succinate and diisotridecyl acelaat, and also diol esters, such as ethylene glycol dioleate, ethylene glycol diisotridecanoate, propylene glycol di(2-ethylhexanoate), propylene glycol diisostearate, propylene glycol dipelargonate, butanediol diisostearate and neopentyl glycol dicaprylate. Esters of C₆-C₂₄ fatty alcohols and/or Guerbet alcohols with aromatic carboxylic acids, saturated and/or unsaturated, especially benzoic acid, esters of C₂-C₁₂ dicarboxylic acids with linear or branched alcohols having from 1 to 22 carbon atoms or polyols having from 2 to 10 carbon atoms and from 2 to 6 hydroxy groups.

### Natural or Synthetic Triglycerides Including Glyceryl Esters and Derivatives

Di- or triglycerides, based on C₆-C₁₈ fatty acids, modified by reaction with other alcohols (caprylic/capric triglyceride, wheat germ glycerides, etc.). Fatty acid esters of polyglycerin (polyglyceryl-n such as polyglyceryl-4 caprate, polyglyceryl-2 isostearate, etc. or castor oil, hydrogenated vegetable oil, sweet almond oil, wheat germ oil, sesame oil, hydrogenated cottonseed oil, coconut oil, avocado oil, corn oil, hydrogenated castor oil, shea butter, cocoa butter, soybean oil, mink oil, sunflower oil, safflower oil, macadamia nut oil, olive oil, hydrogenated tallow, apricot kernel oil, hazelnut oil, borage oil, etc.

Waxes including esters of long-chain acids and alcohols as well as compounds having wax-like properties, e.g., carnauba wax, beeswax (white or yellow), lanolin wax, candelilla wax, ozokerite, japan wax, paraffin wax, microcrystalline wax, ceresin, cetearyl esters wax, synthetic beeswax, etc. Also, hydrophilic waxes as Cetearyl Alcohol or partial glycerides.

### Pearlescent Waxes

Alkylene glycol esters, especially ethylene glycol distearate; fatty acid alkanolamides, especially coco fatty acid diethanolamide; partial glycerides, especially stearic acid monoglyceride; esters of polyvalent, unsubstituted or hydroxy-substituted carboxylic acids with fatty alcohols having from 6 to 22 carbon atoms, especially long-chained esters of tartaric acid; fatty substances, for example fatty alcohols, fatty ketones, fatty aldehydes, fatty ethers and fatty carbonates, which in total have at least 24 carbon atoms, especially lauryl and distearyl ether; fatty acids, such as stearic acid, hydroxystearic acid or behenic acid, ring-opening products of olefin epoxides having from 12 to 22 carbon atoms with fatty alcohols having from 12 to 22 carbon atoms and/or polyols having from 2 to 15 carbon atoms and from 2 to 10 hydroxy groups, and mixtures thereof.

### Hydrocarbon Oils

Mineral oil (light or heavy), petrolatum (yellow or white), microcrystalline wax, paraffinic and isoparaffinic compounds, hydrogenated isoparaffinic molecules as polydecenes and polybutene, hydrogenated polyisobutene, squalane, isohexadecane, isododecane and others from plant and animal kingdom.

### Additional Silicones or Siloxanes (Organosubstituted Polysiloxanes)

Dimethylpolysiloxanes, methylphenylpolysiloxanes, cyclic silicones, and also amino-, fatty acid-, alcohol-, polyether-, epoxy-, fluorine-, glycoside- and/or alkyl-modified silicone compounds, which at room temperature may be in either liquid or resinous form. Linear polysiloxanes, dimethicone (Dow Corning 200 fluid, Rhodia Mirasil DM), dimethiconol, cyclic silicone fluids, cyclopentasiloxanes volatiles (Dow Corning 345 fluid), phenyltrimethicone (Dow Corning 556 fluid). Also suitable are simethicones, which are mixtures of dimethicones having an average chain length of from 200 to 300 dimethylsiloxane units with hydrogenated silicates. A detailed survey by Todd et al. of suitable volatile silicones may in addition be found in Cosm. Toil. 91, 27 (1976).

### Emulsifiers

Any conventionally usable emulsifier can be used for the compositions. Emulsifier systems may comprise for example: carboxylic acids and their salts: alkaline soap of sodium, potassium and ammonium, metallic soap of calcium or magnesium, organic basis soap such as Lauric, palmitic, stearic and oleic acid etc. Alkyl phosphates or phosphoric acid esters, acid phosphate, diethanolamine phosphate, potassium cetyl phosphate. Ethoxylated carboxylic acids or polyethylene glycol esters, PEG-n acylates. Linear fatty alcohols having from 8 to 22 carbon atoms, branched from 2 to 30 mol of ethylene oxide and/or from 0 to 5 mol propylene oxide with fatty acids having from 12 to 22 carbon atoms and with alkylphenols having from 8 to 15 carbon atoms in the alkyl group. Fatty alcohol polyglycol ether such as laureth-n, ceteareth-n, steareth-n, oleth-n. Fatty acid polyglycolether such as PEG-n stearate, PEG-n oleate, PEG-n cocoate. Monoglycerides and polyol esters. C12-C22 fatty acid mono- and di-esters of addition products of from 1 to 30 mol of ethylene oxide with polyols. Fatty acid and polyglycerol ester such as monostearate glycerol, diisostearoyl polyglyceryl-3-diisostearates, polyglyceryl-3-diisostearates, triglyceryl diisostearates, polyglyceryl-2-sesquiisostearates or polyglyceryl dimerates. Mixtures of compounds from a plurality of those substance classes are also suitable. Fatty acid polyglycolesters such as monostearate diethylene glycol, fatty acid and polyethylene glycol esters, fatty acid and saccharose esters such as sucro esters, glycerol and saccharose esters such as sucro glycerides. Sorbitol and sorbitan, sorbitan mono- and di-esters of saturated and unsaturated fatty acids having from 6 to 22 carbon atoms and ethylene oxide addition products. Polysorbate-n series, sorbitan esters such as sesquiisostearate, sorbitan, PEG-(6)-isostearate sorbitan, PEG-(10)-sorbitan laurate, PEG-17-dioleate sorbitan. Glucose derivatives, C8-C22 alkyl-mono and oligo-glycosides and ethoxylated analogues with glucose being preferred as the sugar component. O/W emulsifiers such as methyl gluceth-20 sesquistearate, sorbitan stearate/sucrose cocoate, methyl glucose sesquistearate, cetearyl alcohol/cetearyl glucoside. W/O emulsifiers such as methyl glucose dioleate/methyl glucose isostearate. Sulfates and sulfonated derivatives, dialkylsulfosuccinates, dioctyl succinate, alkyl lauryl sulfonate, linear sulfonated paraffins, sulfonated tetrapropyene sulfonate, sodium lauryl sulfates, ammonium and ethanolamine lauryl sulfates, lauryl ether sulfates, sodium laureth sulfates, sulfosuccinates, acetyl isothionates, alkanolamide sulfates, taurines, methyl taurines, imidazole sulfates. Polysiloxane/polyalkyl/polyether copolymers and derivatives, dimethicone, copolyols, silicone polyethylene oxide copolymer, silicone glycol copolymer. Propoxylated or POE-n ethers (Meroxapols), Polaxamers or poly(oxyethylene)m-block-poly(oxypropylene)n-block(oxyethyl ene). Zwitterionic surfactants that carry at least one quaternary ammonium group and at least one carboxylate and/or sulfonate group in the molecule. Zwitterionic surfactants that are especially suitable are betaines, such as N-alkyl-N,N-dimethylammonium glycinates, cocoalkyldimethylammonium glycinate, N-acylaminopropyl-N,N-dimethylammonium glycinates, cocoacylaminopropyldimethylammonium glycinate and 2-alkyl-3-carboxymethyl-3-hydroxyethylimidazolines each having from 8 to 18 carbon atoms in the alkyl or acyl group and also cocoacylaminoethylhydroxyethylcarboxymethylglycinate, N-alkyl betaine, N-alkylaminobetaines. Alkylimidazolines, alkylopeptides, lipoaminoacides, self-emulsifying bases and the compounds as described in K. F. DePolo, A short textbook of cosmetology, Chapter 8, Table 8-7, p 250-251.

Nonionic bases such as PEG-6 beeswax (and) PEG-6 stearate (and) polyglyceryl-2-isostearate, glyceryl stearate (and) PEG-100 stearate, PEG-5 glyceryl stearate, sorbitan oleate (and) polyglyceryl-3 ricinoleate, sorbitan stearate and sucrose cocoate, glyceryl stearate and laureth-23, cetearyl alcohol and ceteth-20, cetearyl alcohol and polysorbate 60 and PEG-150 and stearate-20, cetearyl alcohol and cetearyl polyglucoside, cetearyl alcohol and ceteareth-20, cetearyl alcohol and PEG-40 castor oil, cetearyl alcohol and PEG-40 castor oil and sodium cetearyl sulfate, stearyl alcohol and steareth-7 and steareth-10, cetearyl alcohol and steareth-7 and steareth-10, glyceryl stearate and PEG-75 stearate, propylene glycol ceteth-3 acetate, propylene glycol isoceth-3 acetate, cetearyl alcohol and ceteth-12 and oleth-12, PEG-6 stearate and PEG-32 stearate, PEG-6 stearate and ceteth-20 and steareth-20, PEG-6 stearate and ceteth- and glyceryl stearate and steareth-20, glyceryl stearate and ceteareth-20.

Anionic alkaline bases such as PEG-2 stearate SE, glyceryl stearate SE, propylene glycol stearate. Anionic acid bases such as cetearyl Alcohol and Sodium cetearyl sulfate, cetearyl alcohol and sodium lauryl sulfate, trilaneth-4 phosphate and glycol stearate and PEG-2 stearate, glyceryl stearate and sodium lauryl Sulfate. Cationic acid bases such as cetearyl alcohol and cetrimonium bromide.

Suitable emulsifiers may also include, without limitations, PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, Glyceryl Stearate (and) PEG-100 Stearate, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate, Steareth-2, Steareth-12, Oleth-2, Ceteth-2, Laureth-4, Oleth-Oleth-10/Polyoxyl 10 Oleyl Ether, Ceteth-10, lsosteareth-20, Ceteareth-20, Oleth-20, Steareth-20, Steareth-21, Ceteth-20, lsoceteth-20, Laureth-23, Steareth-100, Glyceryl Stearate Citrate, Glyceryl Stearate SE (self-emulsifying), stearic acid, salts of stearic acid, polyglyceryl-3-methylglycosedistearate, or a combination thereof.

Further suitable emulsifiers are phosphate esters and the salts thereof such as cetyl phosphate (Amphisol® A), diethanolamine cetyl phosphate (Amphisol® DEA), potassium cetyl phosphate (Amphisol® K), sodium cetearyl sulfate, sodium glyceryl oleate phosphate, hydrogenated vegetable glycerides phosphate and mixtures thereof. Further suitable emulsifiers are sorbitan oleate, sorbitan sesquioleate, sorbitan isostearate, sorbitan trioleate, Cetearyl Glucoside, Lauryl Glucoside, Decyl Glucoside, Sodium Stearoyl Glutamate, Sucrose Polystearate and Hydrated Polyisobutene. Furthermore, one or more synthetic polymers may be used as an emulsifier. For example, PVP eicosene copolymer, acrylates/C₁₀₋₃₀ alkyl acrylate crosspolymer, acrylates/steareth-20 methacrylate copolymer, PEG-22/dodecyl glycol copolymer, PEG-45/dodecyl glycol copolymer, and mixtures thereof.

### Adjuvants and Additives

The cosmetic sunscreen compositions, for example creams, gels, lotions, alcoholic and aqueous/alcoholic solutions, emulsions, wax/fat compositions, stick preparations, powders or ointments, may in addition contain, as further adjuvants and additives such as, mild surfactants, super-fatting agents, consistency regulators, thickeners, polymers, stabilizers, biogenic active ingredients, swelling agents, further UV light-protective factors, antioxidants, hydrotropic agents, preservatives, self-tanning agents, solubilizers, perfume oils, colorants, bacteria-inhibiting agents and the like.

### Super-Fatting Agents

Substances suitable for use as super-fatting agents are, for example, lanolin and lecithin and also polyethoxylated or acetylated lanolin and lecithin derivatives, polyol fatty acid esters, monoglycerides and fatty acid alkanolamides, the latter simultaneously acting as foam stabilizers.

### Surfactants

Examples of suitable mild surfactants, that is to say surfactants especially well tolerated by the skin, include fatty alcohol polyglycol ether sulfates, monoglyceride sulfates, mono- and/or di-alkyl sulfosuccinates, fatty acid isethionates, fatty acid sarcosinates, fatty acid taurides, fatty acid glutamates, .alpha.-olefin sulfonates, ethercarboxylic acids, alkyl oligoglucosides, fatty acid glucamides, alkylamidobetaines and/or protein fatty acid condensation products, the latter preferably being based on wheat proteins.

### Additional Consistency Regulators/Thickeners and Rheology Modifiers

Silicium dioxide, magnesium silicates, aluminium silicates, polysaccharides or derivatives thereof for example hyaluronic acid, xanthan gum, guar-guar, agar-agar, alginates, carrageenan, gellan, pectines, or modified cellulose such as hydroxycellulose, hydroxypropylmethylcellulose. In addition polyacrylates or homopolymer of reticulated acrylic acids and polyacrylamides, carbomer (CARBOPOL types 980, 981, 1382, ETD 2001, ETD2020, ULTREZ10) or SALCARE range such as SALCARE SC80 (steareth-10 allyl ether/acrylates copolymer), Salcare SC81 (acrylates copolymer), Salcare SC91 and Salcare AST (sodium acrylates copolymer/PPG-1 trideceth-6), SEPIGEL 305 (polyacrylamide/laureth-7), SIMULGEL NS and SIMULGEL EG (hydroxyethyl acrylate/sodium acryloyldimethyl taurate copolymer), STABILEN 30 (acrylates/vinyl isodecanoate crosspolymer), PEMULEN TR-1 (acrylates/C10-30 alkyl acrylate crosspolymer), LUVIGEL EM (sodium acrylates copolymer), ACULYN 28 (acrylates/beheneth-25 methacrylate copolymer), etc.

### Polymers

As anionic, zwitterionic, amphoteric and non-ionic polymers there come into consideration, for example, vinyl acetate/crotonic acid copolymers, vinylpyrrolidone/vinyl acrylate copolymers, vinyl acetate/butyl maleate/isobornyl acrylate copolymers, methyl vinyl ether/maleic anhydride copolymers and esters thereof, uncrosslinked polyacrylic acids and polyacrylic acids crosslinked with polyols, acrylamidopropyl-trimethylammonium chloride/acrylate copolymers, octyl acrylamide/methyl methacrylate-tert-butylaminoethyl methacrylate/2-hydroxypropyl methacrylate copolymers, polyvinylpyrrolidone, vinylpyrrolidone/vinyl acetate copolymers, vinylpyrrolidone/dimethylaminoethyl methacrylate/vinyl caprolactam terpolymers and also optionally derivatized cellulose ethers and silicones. Furthermore, the polymers as described in EP 1093796 (pages 3-8, paragraphs 17-68) may be used.

### Biogenic Active Ingredients

Biogenic active ingredients are to be understood as meaning, for example tocopherol palmitate, deoxyribonucleic acid, retinol, bisabolol, phytantriol, panthenol, AHA acids, amino acids, ceramides, pseudoceramides, essential oils, plant extracts and vitamin complexes.

### Additional Antioxidants

In addition to the primary light-protective substances it is also possible to use secondary light-protective substances of the antioxidant kind that interrupt the photochemical reaction chain triggered when UV radiation penetrates the skin or hair. Typical examples of such antioxidants are amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotinoids, carotenes, lycopene and derivatives thereof, chlorogenic acid and derivatives thereof, lipoic acid and derivatives thereof (e.g. dihydrolipoic acid), aurothioglycose, propylthiouracil and other thiols (e.g. thioredoxin, glutathione, cysteine, cystine, cystamine and the glycosyl, N-acetyl, methyl, ethyl, propyl, amyl, butyl, lauryl, palmitoyl, oleyl, linoleyl, cholesteryl and glyceryl esters thereof) and al so salts thereof, dilauryl thiodipropionate, di stearyl thiodipropionate, thiodipropionic acid and derivatives thereof (esters, ethers, peptides, lipids, nucleotides, nucleosides and salts) and also sulfoximine compounds (e.g. buthionine sulfoximines, homocysteine sulfoximine, buthionine sulfones, penta-, hexa-, hepta-thionine sulfoximine), also (metal) chelating agents (e.g. hydroxy fatty acids, palmitic acid phytic acid, lactoferrin), hydroxy acids (e.g. citric acid, lactic acid, malic acid), humic acid, bile acid, bile extracts, bilirubin, biliverdin, EDTA, EDDS, EGTA and derivatives thereof, unsaturated fatty acids and derivatives thereof (e.g. linolenic acid, linoleic acid, oleic acid), folic acid and derivatives thereof, ubiquinone and ubiquinol and derivatives thereof, vitamin C and derivatives (e.g. ascorbyl palmitate, magnesium ascorbyl phosphate, ascorbyl acetate), tocopherols and derivatives (e.g. vitamin E acetate), vitamin A and derivatives (e.g. vitamin A palmitate) and also coniferyl benzoate of benzoin resin, rutinic acid and derivatives thereof, glycosylrutin, ferulic acid, furfurylidene glucitol, carnosine, butyl hydroxytoluene, butyl hydroxyanisole, nordihydroguaiaretic acid, trihydroxybutyrophenone, uric acid and derivatives thereof, mannose and derivatives thereof, superoxide dismutase, N-[3-(3,5-di-tert-butyl-4-hydroxyphenyl)propionyl]sulfanilic acid (and salts thereof, for example the disodium salts), selenium and derivatives thereof (e.g. selenium methionine), stilbene and derivatives thereof (e.g. stilbene oxide, trans-stilbene oxide) and the derivatives suitable according to the invention (salts, esters, ethers, sugars, nucleotides, nucleosides, peptides and lipids) of those mentioned active ingredients. HALS(=“Hindered Amine Light Stabilizers”) compounds may also be mentioned.

### Hydrotropic Agents

To improve the flow behavior it is also possible to employ hydrotropic agents, for example ethoxylated or non-ethoxylated mono-alcohols, diols or polyols with a low number of carbon atoms or their ethers (e.g. ethanol, isopropanol, 1,2-dipropanediol, propylene glycol, glycerin, ethylene glycol, ethylene glycol monoethylether, ethylene glycol monobutylether, propylene glycol monomethylether, propylene glycol monoethylether, propylene glycol monobutylether, diethylene glycol monomethylether; diethylene glycol monoethylether, diethylene glycol monobutylether and similar products). The polyols that come into consideration for that purpose have preferably from 2 to 15 carbon atoms and at least two hydroxy groups. The polyols may also contain further functional groups, especially amino groups, and/or may be modified with nitrogen. Typical examples are as follows: glycerol, alkylene glycols, for example ethylene glycol, diethylene glycol, propylene glycol, butylene glycol, hexylene glycol and also polyethylene glycols having an average molecular weight of from 100 to 1000 Dalton; technical oligoglycerol mixtures having an intrinsic degree of condensation of from 1.5 to 10, for example technical diglycerol mixtures having a diglycerol content of from 40 to 50% by weight; methylol compounds, such as, especially, trimethylolethane, trimethylolpropane, trimethylolbutane, pentaerythritol and dipentaerythritol; lower alkyl-glucosides, especially those having from 1 to 8 carbon atoms in the alkyl radical, for example methyl and butyl glucoside; sugar alcohols having from 5 to 12 carbon atoms, for example sorbitol or mannitol; sugars having from 5 to 12 carbon atoms, for example glucose or saccharose; amino sugars, for example glucamine; dialcohol amines, such as diethanolamine or 2-amino-1,3-propanediol.

### Additional Preservatives and Bacteria-Inhibiting Agents

Suitable preservatives include, for example, Methyl-, Ethyl-, Propyl-, Butyl-parabens, Benzalkonium chloride, 2-Bromo-2-nitro-propane-1,3-diol, Dehydroacetic acid, Diazolidinyl Urea, 2-Dichloro-benzyl alcohol, DMDM hydantoin, Formaldehyde solution, Methyldibromoglutanitrile, Phenoxyethanol, Sodium Hydroxymethylglycinate, Imidazolidinyl Urea, Triclosan and further substance classes listed in the following reference: K. F. DePolo-A short textbook of cosmetology, Chapter 7, Table 7-2, 7-3, 7-4 and 7-5, p 210-219.

In certain embodiments, the addition of a preservative is not necessary, presumably, without being construed as limiting, due to low water activity and an environment not conducive to microbial growth. Hence, in certain embodiments, the topical composition is free or substantially free of a preservative.

### Bacteria-Inhibiting Agents

Typical examples of bacteria-inhibiting agents are preservatives that have a specific action against gram-positive bacteria, such as 2,4,4′-trichloro-2′-hydroxydiphenyl ether, chlorhexidine (1,6-di(4-chlorophenyl-biguanido)hexane) or TCC (3,4,4′-trichlorocarbanilide). A large number of aromatic substances and ethereal oils also have antimicrobial properties. Typical examples are the active ingredients eugenol, menthol and thymol in clove oil, mint oil and thyme oil. A natural deodorizing agent of interest is the terpene alcohol farnesol (3,7,11-trimethyl-2,6,10-dodecatrien-1-ol), which is present in lime blossom oil. Glycerol monolaurate has also proved to be a bacteriostatic agent.

### Colorants

There may be used as colorants the substances that are suitable and permitted for cosmetic purposes, as compiled, for example, in the publication “Kosmetische Farbemittel” of the Farbstoffkommission der Deutschen Forschungsgemeinschaft, Verlag Chemie, Weinheim, 1984, pages 81 to 106.

### UV Screening Agents

Suitable sun screening agents would include a range of organic UV screening agents selected from the group consisting of 1(+/−)-1,7,7-trimethyl-3-[(4-methylphenyl)methylene]bicyclo-[2.2.1]heptan-2-one; p-methyl benzylidene camphor, 1,7,7-trimethyl-3-(phenylmethylene)bicyclo[2.2.1]heptan-2-one; benzylidene camphor, (2-Hydroxy-4-methoxyphenyl)(4-methylphenyl)methanone, 2,4-dihydroxybenzophenone, 2,2′,4,4′-tetrahydroxybenzophenone, 2-Hydroxy-4-methoxy benzophenone, 2-Hydroxy-4-methoxy benzophenone-5-sulfonic acid, 2,2′-dihydroxy-4,4′-dimethoxybenzophenone, 2,2′-Dihydroxy-4-methoxybenzophenone, Alpha-(2-oxoborn-3-ylidene)toluene-4-sulphonic acid and its salts, 1-[4-(1,1-dimethylethyl)phenyl]-3-(4-methoxyphenyl)propane-1,3-dione, Methyl N,N,N-trimethyl-4-[(4,7,7-trimethyl-3-oxobicyclo[2,2,1]-hept-2-ylidene)methyl]anilinium sulphate, 3,3,5-Trimethyl cyclohexyl-2-hydroxy benzoate, Isopentyl p-methoxycinnamate, Menthyl-o-aminobenzoate, 2-Ethylhexyl 2-cyano, 3,3-diphenylacrylate, 2-ethylhexyl 4-(dimethylamino)benzoate, 2-ethylhexyl 4-methoxycinnamate, 2-ethylhexyl salicylate, Benzoic acid, 4,4′,4″-(1,3,5-triazine-2,4,6-triyltriimino)tris-, tris(2-ethylhexyl)ester, 4-aminobenzoic acid, Benzoic acid, 4-amino-, ethyl ester, polymer with oxirane, 2-phenyl-1H-benzimidazole-5-sulphonic acid, 2-Propenamide, N-[[4-[(4,7,7-trimethyl-3-oxobicyclo[2.2.1]hept-2-ylidene)methyl]phenyl]methyl]-, homopolymer. Triethanolamine salicylate, 3,3′-(1,4-phenylenedimethylene)bis[7,7-dimethyl-2-oxo-bicyclo[2.2.1]heptane-1 methanesulfonic acid], Titanium dioxide, 2,2′-Methylene-bis-[6-(2H-benzotriazol-2-yl)-4-(1,1,3,3-tetramethylbutyl)-phenol], Bis-Ethylhexyloxyphenol Methoxyphenyl Triazine,1H-Benzimidazole-4,6-disulfonic acid, 2,2′-(1,4-phenylene)bis-, disodium salt, Benzoic acid, 4,4′-[[6-[[4-[[(1,1-dimethylethyl)amino]carbonyl]-phenyl]amino]1,3,5-triazine-2,4-diyl]diimino]bis-, Phenol, 2-(2H-benzotriazol-2-yl)-4-methyl-6-[2-methyl-3-[1,3,3,3-tetramethyl-1-[(trimethylsily)oxy]disiloxanyl] propyl]-, Dimethicodiethylbenzalmalonate, Benzenesulfonic acid, 3-(2H-benzotriazol-2-yl)-4-hydroxy-5-(1-methyl propyl)-, monosodium salt, Benzoic acid, 2-[4-(diethylamino)-2-hydroxybenzoyl]-, hexyl ester, 1-Dodecanaminium, N-[3-[[4-(dimethylamino)benzoyl]amino]-propyl] N,N-dimethyl-, salt with 4-methylbenzenesulfonic acid (1:1), 1-Propanaminium, N,N,N-trimethyl-3-[(1-oxo-3-phenyl-2-propenyl) amino]-, chloride, 1H-Benzimidazole-4,6-disulfonic acid, 2,2′-(1,4-phenylene)bis-, 1,3,5-Triazine, 2,4,6-tris(4-methoxyphenyl)-,1,3,5-Triazine, 2,4,6-tris[4-[(2-ethylhexyl)oxy]phenyl]-, 1-Propanaminium, 3-[[3-[3-(2H-benzotriazol-2-yl)-5-(1,1-dimethylethyl)-4-hydroxyphenyl]-1-oxopropyl]amino]-N,N-diethyl-N-methyl-, methyl sulfate (salt), 2-Propenoic acid, 3-(1H-imidazol-4-yl)-, Benzoic acid, 2-hydroxy-, [4-(1-methylethyl)phenyl]methyl ester, 1,2,3-Propanetriol, 1-(4-aminobenzoate), Benzeneacetic acid, 3,4-dimethoxy-a-oxo-, 2-Propenoic acid, 2-cyano-3,3-diphenyl-, ethyl ester, Anthralinic acid, p-menth-3-yl ester, 2,2′-bis(1,4-phenylene)-1H-benzimidazole-4,6-disulphonic acid mono sodium salt or Disodium phenyl dibenzimidazole tetrasulfonate, 1,3,5-Triazine-2,4,6-triamine and N,N′-bis[4-[5-(1,1-dimethylpropyl)-2-benzoxazolyl]phenyl]-N″-(2-ethylhexyl).

Agents that Absorb UV Light and Provide Photo Protection to the Skin

Suitable agents that absorb UV light, provide photo protection to the skin, and optionally provide sunless tanning may include a compound of formula I:

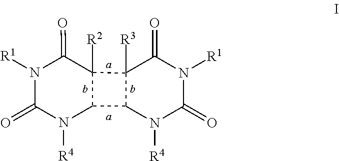

wherein:
each R¹ is independently H, (C₁-C₆)alkyl, (C₃-C₇)carbocycle or RₐC(═O)—, and the two R⁴ groups together form a —(C₃-C₈)alkyl-group, a —(C₂-C₆)alkyl-Y—(C₂-C₆)alkyl-group or a —(C₁-C₆)alkyl-Y′—(C₁-C₆)alkyl-group; or
each R⁴ is independently H, (C₁-C₆)alkyl, (C₃-C₇)carbocycle or RₐC(═O)—, and the two R¹ groups together form a —(C₃-C₈)alkyl-group, a —(C₂-C₆)alkyl-Y—(C₂-C₆)alkyl-group or a —(C₁-C₆)alkyl-Y′—(C₁-C₆)alkyl-group; or
the two R⁴ groups together form a —(C₃-C₈)alkyl-group, a —(C₂-C₆)alkyl-Y—(C₂-C₆)alkyl group or a —(C₁-C₆)alkyl-Y′—(C₁-C₆)alkyl-group and the two R¹ groups together form a —(C₃-C₈)alkyl-group, a —(C₂-C₆)alkyl-Y—(C₂-C₆)alkyl-group or a —(C₁-C₆)alkyl-Y′—(C₁-C₆)alkyl-group;
the dashed bonds labeled “a” are absent and the dashed bonds labeled “b” are double bonds; or all the dashed bonds are single bonds;
R² is H, (C₁-C₆)alkyl or aryl, wherein aryl is optionally substituted with one or more Z¹ groups;
R³ is H, (C₁-C₆)alkyl or aryl, wherein aryl is optionally substituted with one or more Z¹ groups;
Y is O, S, NH, NR C, P, P(═O) or POH;
Y′ is Si(R_{b})₂ or —Si(R_{b})₂—O—Si(R_{b})₂—;
each Rₐ is independently (C₁-C₆)alkyl, (C₃-C₇)carbocycle or aryl, wherein aryl is optionally substituted with one or more Z¹ groups;
each R_{b} is independently (C₁-C₆)alkyl, (C₃-C₇)carbocycle or aryl, wherein aryl is optionally substituted with one or more Z¹ groups;
each R_{c} is independently R_{g} or a C₁-C₁₈ saturated or unsaturated carbon chain that is optionally substituted with one or more groups independently selected from oxo (═O), hydroxy, mercapto, (C₁-C₆)alkoxy, (C₁-C₆)alkoxycarbonyl, (C₁-C₆)alkanoyloxy, NR_{d}Rₑ, carboxy, and aryl, wherein any aryl of Rₑ is optionally substituted with one or more R_{f};
each R_{d} and Rₑ is independently selected from H, (C₁-C₆)alkyl, (C₁-C₆)alkanoyl, phenyl, benzyl, and R_{g};
each R_{f} is independently selected from (C₁-C₆)alkyl, (C₁-C₆)alkoxy, (C₁-C₆)alkoxycarbonyl, (C₁-C₆)alkanoyloxy, —C(═O)-phenyl, and —C(═O)CH₂C(═O)-phenyl, wherein any phenyl is optionally substituted with one or more groups independently selected from (C₁-C₆)alkyl, SO₃H, and (C₁-C₆)alkoxy;
each R_{g} is

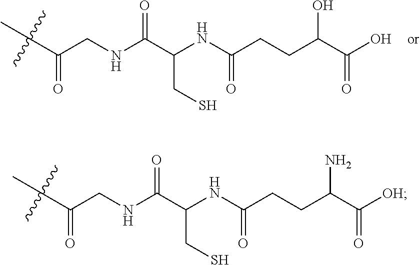 [SMILES: *C(=O)CNC(=O)C(CS)NC(=O)CCC(O)C(=O)O ; *C(=O)CNC(=O)C(CS)NC(=O)CCC(N)C(=O)O]

each Z¹ is independently selected from (C₁-C₆)alkyl, halogen, —CN, —ORₙ₁, —NR_{g1}Rᵣ₁, —NRₙ₁CORₚ₁, —NRₙ₁CO₂Rₚ₁, NO₂, —C(O)Rₙ₁, —C(O)ORₙ₁ and —C(O)NR_{q1}Rᵣ₁, wherein any (C₁-C₆)alkyl of Z¹ is optionally substituted with one or more (e.g. 1, 2, 3, 4, 5 or 6) halogen;
each Rₙ₁ is independently selected from H and (C₁-C₆)alkyl, wherein any (C₁-C₆)alkyl of Rₙ₁ is optionally substituted with one or more (e.g. 1, 2, 3, 4, 5 or 6) halogen;
each Rₚ₁ is independently (C₁-C₆)alkyl; and
R_{q1} and Rᵣ₁ are each independently selected from H and (C₁-C₆)alkyl or R_{q1} and Rᵣ₁ together with the nitrogen to which they are attached form a piperidine, pyrrolidine, morpholine, azetidine, thiomorpholine, piperazine or 4-methylpiperazine;
or a salt thereof.

Specific groups of compounds and specific compounds of Formula I that may be incorporated in the topical composition described herein and methods of preparing such compounds are described in U.S. Pat. No. 9,403,778 and in U.S. Pat. No. 9,987,211, which are incorporated herein by reference in its entirety.

Suitable agents that absorb UV light, provide photo protection to the skin, and optionally provide sunless tanning may include a compound of formula II:

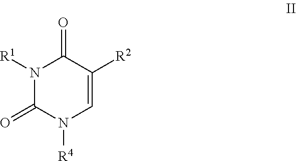

or a salt thereof, wherein:
R¹ is H, (C₁-C₆)alkyl, (C₃-C₇)carbocycle or RₐC(═O)—;
R² is H, (C₁-C₆)alkyl or aryl, wherein aryl is optionally substituted with one or more (e.g. 1, 2, 3, 4 or 5) Z¹ groups;
R⁴ is H, (C₃-C₇)carbocycle or RₐC(═O)—;
Rₐ is or (C₁-C₆)alkyl, (C₃-C₇)carbocycle or aryl, wherein aryl is optionally substituted with one or more (e.g. 1, 2, 3, 4 or 5) Z¹ groups;
each Z¹ is independently selected from (C₁-C₆)alkyl, halogen, —CN, —ORₙ₁, —NR_{g1}Rᵣ₁, —NRₙ₁CORₚ₁, —NRₙ₁CO₂Rₚ₁, NO₂, —C(O)Rₙ₁, —C(O)ORₙ₁ and —C(O)NR_{q1}Rᵣ₁, wherein any (C₁-C₆)alkyl of Z¹ is optionally substituted with one or more (e.g. 1, 2, 3, 4, 5 or 6) halogen; each Rₙ₁ is independently selected from H and (C₁-C₆)alkyl, wherein any (C₁-C₆)alkyl of Rₙ₁ is optionally substituted with one or more (e.g. 1, 2, 3, 4, 5 or 6) halogen; each Rₚ₁ is independently (C₁-C₆)alkyl; and R_{q1} and Rᵣ₁ are each independently selected from H and (C₁-C₆)alkyl or R_{q1} and Rᵣ₁ together with the nitrogen to which they are attached form a piperidine, pyrrolidine, morpholine, azetidine, thiomorpholine, piperazine or 4-methylpiperazine.

Specific groups of compounds and specific compounds of Formula II that may be incorporated in the topical composition described herein and methods of preparing such compounds are described in U.S. Pat. No. 9,987,211, which is incorporated herein by reference in its entirety.

Suitable agents that absorb UV light and provide photo protection to the skin may include a compound of formula III:

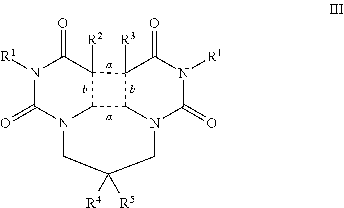

wherein:
each R¹ is independently H, (C₁-C₆)alkyl, (C₃-C₇)carbocycle or RₐC(═O; or the two R¹ groups together form a —(C₃-C₈)alkyl-group, a —(C₂-C₆)alkyl-Y—(C₂-C₆)alkyl-group or a —(C₁-C₆)alkyl-Y′—(C₁-C₆)alkyl-group; or
the dashed bonds labeled “a” are absent and the dashed bonds labeled “b” are double bonds; or all the dashed bonds are single bonds;
R² is H, (C₁-C₆)alkyl or aryl, wherein aryl is optionally substituted with one or more (e.g. 1, 2, 3, 4 or 5) Z¹ groups;
R³ is H, (C₁-C₆)alkyl or aryl, wherein aryl is optionally substituted with one or more (e.g. 1, 2, 3, 4 or 5) Z¹ groups;
R⁴ is hydroxy, carboxy, (C₁-C₆)alkoxycarbonyl, —OPO₃H₂, —OR_{c}, or —NR_{d}Rₑ; and R⁵ is H; or
R⁴ and R⁵ taken together are oxo;
Y is O, S, NH, P, P(═O) or POH;
Y′ is Si(R_{b})₂ or —Si(R_{b})₂—O—Si(R_{b})₂—;
each Rₐ is independently (C₁-C₆)alkyl, (C₃-C₇)carbocycle or aryl, wherein aryl is optionally substituted with one or more (e.g. 1, 2, 3, 4 or 5) Z¹ groups;
each R_{b} is independently (C₁-C₆)alkyl, (C₃-C₇)carbocycle or aryl, wherein aryl is optionally substituted with one or more (e.g. 1, 2, 3, 4 or 5) Z¹ groups;
R_{c} is R_{f} or a C₁-C₂₀ saturated or C₂-C₂₀ unsaturated carbon chain that is optionally substituted with one or more groups independently selected from oxo (═O), hydroxy, mercapto, (C₁-C₆)alkoxy, (C₁-C₆)alkoxycarbonyl, (C₁-C₆)alkanoyloxy, NR_{d}Rₑ, carboxy, and aryl, wherein aryl is optionally substituted with one or more (e.g. 1, 2, 3, 4 or 5) Z¹ groups;
R_{d} is H, (C₁-C₆)alkyl, or (C₁-C₆)alkanoyl;
Rₑ is H or a C₁-C₂₀ saturated or C₂-C₂₀ unsaturated carbon chain that is optionally substituted with one or more groups independently selected from oxo (═O), hydroxy, mercapto, (C₁-C₆)alkoxy, (C₁-C₆)alkoxycarbonyl, (C₁-C₆)alkanoyloxy, NR_{d}Rₑ, carboxy, and aryl, wherein aryl is optionally substituted with one or more (e.g. 1, 2, 3, 4 or 5) Z¹ groups;
each R_{f}, is:

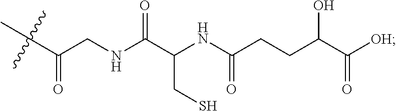 [SMILES: *C(=O)CNC(=O)C(CS)NC(=O)CCC(O)C(=O)O]

each Z¹ is independently selected from (C₁-C₆)alkyl, halogen, —CN, —ORₙ₁, —NR_{g1}Rᵣ₁, —NRₙ₁CORₚ₁, —NRₙ₁CO₂Rₚ₁, NO₂, —C(O)Rₙ₁, —C(O)ORₙ₁ and —C(O)NR_{q1}Rᵣ₁, wherein any (C₁-C₆)alkyl of Z¹ is optionally substituted with one or more (e.g. 1, 2, 3, 4, 5 or 6) halogen;
each Rₙ₁ is independently selected from H and (C₁-C₆)alkyl, wherein any (C₁-C₆)alkyl of Rₙ₁ is optionally substituted with one or more (e.g. 1, 2, 3, 4, 5 or 6) halogen;
each Rₚ₁ is independently (C₁-C₆)alkyl; and
R_{q1} and Rᵣ₁ are each independently selected from H and (C₁-C₆)alkyl or R_{q1} and Rᵣ₁ together with the nitrogen to which they are attached form a piperidine, pyrrolidine, morpholine, azetidine, thiomorpholine, piperazine or 4-methylpiperazine;
or a salt thereof.

Specific groups of compounds and specific compounds of Formula III that may be incorporated in the topical composition described herein and methods of preparing such compounds are described in U.S. Pat. No. 9,364,406 and in U.S. Pat. No. 9,987,211, which are incorporated herein by reference in its entirety.

### Sunscreening Agents

Topical Compositions disclosed herein may include sun screening agents such as avobenzone, ecamsule, methyl anthranilate, oxybenzone, dioxybenzone, sulisobenzone, octinoxate, homosalate, octocrylene and octisalate. Such compositions may comprise organic UV filters, so-called hydrophilic or lipophilic sun-protection filters, which are effective in the UVA region and/or UVB region and (/or IR and/or VIS region (absorbers). These substances can be selected, in particular, from cinnamic acid derivatives, salicylic acid derivatives, camphor derivatives, triazine derivatives, β,β-diphenylacrylate derivatives, p-aminobenzoic acid derivatives and polymeric filters and silicone filters, which are described in the WO93/04665. Further examples of organic filters are indicated in patent application EP-A 0 487 404. Particularly suitable for a combination are: para-aminobenzoic acid and derivatives thereof: PABA, Ethyl PABA, Ethyl dihydroxypropyl PABA, Ethylhexyl dimethyl PABA, for example marketed by ISP under the name “Escalol 507”, Glyceryl PABA, PEG-25 PABA, for example marketed under the name “Uvinul P25” by BASF.

Other UV filter ingredients which may be incorporated in the topical compositions of the disclosure include:

Salicylates: Homosalate marketed by Merck under the name “Eusolex HMS”; Ethylhexyl salicylate, for example marketed by Symrise under the name “Neo Heliopan OS”, Dipropylene glycol salicylate, for example marketed by Scher under the name “Dipsal”, TEA salicylate, for example marketed by Symrise under the name “Neo Heliopan TS”.

β,β-Diphenylacrylate derivatives: Octocrylene, for example marketed by Merck under the name “Eusolex® OCR”, “Uvinul N539” from BASF, Octocrylene, for example marketed by BASF under the name “Uvinul N35”.

Benzophenone derivatives: Benzophenone-1, for example marketed under the name “Uvinul 400”; Benzophenone-2, for example marketed under the name “Uvinul D50”; Benzophenone-3 or Oxybenzone, for example marketed under the name “Uvinul M40”; Benzophenone-4, for example marketed under the name “Uvinul MS40”; Benzophenone-9, for example marketed by BASF under the name “Uvinul DS-49”, Benzophenone-5, Benzophenone-6, for example marketed by Norquay under the name “Helisorb 11”, Benzophenone-8, for example marketed by American Cyanamid under the name “Spectra-Sorb UV-24”, Benzophenone-12 n-hexyl 2-(4-diethylamino-2-hydroxybenzoyl)benzoate or 2-hydroxy-4-methoxybenzophenone, marketed by Merck, Darmstadt, under the name Eusolex® 4360.

Benzylidenecamphor derivatives: 3-Benzylidenecamphor, for example marketed by Chimex under the name “Mexoryl SD”, 4-Methylbenzylidenecamphor, for example marketed by Merck under the name “Eusolex 6300”, benzylidenecamphorsulfonic acid, for example marketed by Chimex under the name “Mexoryl SL”, Camphor benzalkonium methosulfate, for example marketed by Chimex under the name “Mexoryl SO”, terephthalylidenedicamphorsulfonic acid, for example marketed by Chimex under the name “Mexoryl SX”, Polyacrylamidomethylbenzylidenecamphor marketed by Chimex under the name “Mexoryl SW”.

Phenylbenzimidazole derivatives: phenylbenzimidazolesulfonic acid, for example marketed by Merck under the name “Eusolex 232”, disodium phenyl dibenzimidazole tetrasulfonate, for example marketed by Symrise under the name “Neo Heliopan AP”.

Phenylbenzotriazole derivatives: Drometrizole trisiloxane, for example marketed by Rhodia Chimie under the name “Silatrizole”,

Methylenebis(benzotriazolyl)tetramethylbutylphenol in solid form, for example marketed by Fairmount Chemical under the name “MIXXIM BB/100”, or in micronized form as an aqueous dispersion, for example marketed by BASF under the name “Tinosorb M”.

Triazine derivatives: ethylhexyltriazone, for example marketed under the name “Uvinul T150” by BASF, diethylhexylbutamidotriazone, for example marketed under the name “Uvasorb HEB” by Sigma 3V, 2,4,6-tris(diisobutyl 4′-aminobenzalmalonate)-s-triazine or 2,4,6-tris(biphenyl)-1,3,5-triazine. marketed as Tinosorb A2B by BASF, 2,2′-[6-(4-methoxyphenyl)-1,3,5-triazine-2,4-diyl]bis[5-(2-ethylhexyl)oxy]phenol, marketed as Tinosorb S by BASF, N2,N4-bis[4-[5-(1,1-dimethylpropyl)-2-benzoxazolyl]phenyl]-N-6-(2-ethylhexyl)-1,3,5-triazine-2,4,6-triamine marketed as Uvasorb K 2A by Sigma 3V.

Anthraniline derivatives: Menthyl anthranilate, for example marketed by Symrise under the name “Neo Heliopan MA”.

Imidazole derivatives: Ethylhexyldimethoxybenzylidenedioxoimidazoline propionate.

Benzalmalonate derivatives: polyorganosiloxanes containing functional benzalmalonate groups, such as, for example, polysilicone-15, for example marketed by Hoffmann LaRoche under the name “Parsol SLX”.

4,4-Diarylbutadiene derivatives: 1,1-Dicarboxy(2,2′-dimethylpropyl)-4,4-diphenylbutadiene.

Benzoxazole derivatives: 2,4-bis[5-(1-dimethylpropyl)benzoxazol-2-yl(4-phenyl) imino]-6-(2-ethylhexyl)imino-1,3,5-triazine, for example marketed by Sigma 3V under the name Uvasorb K2A, and mixtures comprising this.

Suitable organic UV-protecting substances can preferably be selected from: Ethylhexyl salicylate, Phenylbenzimidazolesulfonic acid, Benzophenone-3, Benzophenone-4, Benzophenone-5, n-Hexyl 2-(4-diethylamino-2-hydroxybenzoyl)benzoate, 4-Methylbenzylidenecamphor, Terephthalylidenedicamphorsulfonic acid, Disodium phenyldibenzimidazoletetrasulfonate, Methylenebis(benzotriazolyl)tetramethylbutylphenol, Ethylhexyl Triazone, Diethylhexyl Butamido Triazone, Drometrizole trisiloxane, Polysilicone-15,1,1-Dicarboxy(2,2′-dimethylpropyl)-4,4-diphenylbutadiene, 2,4-bis[5-1 (dimethylpropyl)benzoxazol-2-yl(4-phenyl) imino]-6-(2-ethylhexyl)imino-1,3,5-triazine and mixtures thereof.

The compositions of the invention may comprise further inorganic UV filters, so-called particulate UV filters. These combinations with particulate UV filters are possible both as powder and also as dispersion or paste. In an embodiment the inorganic UV filter is a titanium dioxide, such as, for example, coated titanium dioxide (for example Eusolex® T-2000, Eusolex® T-AQUA, Eusolex® T-AVO, Eusolex® T-OLEO), a zinc oxide (for example Sachtotec), an iron oxide or a cerium oxide and/or zirconium oxide. Furthermore, combinations with pigmentary titanium dioxide or zinc oxide are also possible, where the particle size of these pigments are greater than or equal to 200 nm, for example Hombitan® FG or Hombitan® FFPharma.

Compositions of the invention may comprise inorganic UV filters which have been after treated by conventional methods, as described, for example, in Cosmetics & Toiletries, 1990, 105, 53-64. One or more of the following aftertreatment components can be: amino acids, beeswax, fatty acids, fatty acid alcohols, anionic surfactants, lecithin, phospholipids, sodium, potassium, zinc, iron or aluminium salts of fatty acids, polyethylenes, silicones, proteins (particularly collagen or elastin), alkanolamines, silicon dioxide, aluminium oxide, further metal oxides, phosphates, such as sodium hexametaphosphate, or glycerin.

In an embodiment, particulate UV filters used in compositions of the invention are:
untreated titanium dioxides, such as, the products Microtitanium Dioxide MT 500 B from Tayca; titanium dioxide P25 from Degussa,
after treated micronized titanium dioxides with aluminium oxide and silicone dioxide aftertreatment, such as, the product “Microtitanium Dioxide MT 100 SA from Tayca; or the product “Tioveil Fin” from Uniqema,
after treated micronized titanium dioxides with aluminium oxide and/or aluminium stearate/laurate aftertreatment, such as, Microtitanium Dioxide MT 100 T from Tayca, Eusolex T-2000 from Merck,
after treated micronized titanium dioxides with iron oxide and/or iron stearate aftertreatment, such as, the product “Microtitanium Dioxide MT 100 F” from Tayca,
after treated micronized titanium dioxides with silicone dioxide, aluminium oxide and silicone aftertreatment, such as, the product “Microtitanium Dioxide MT 100 SAS”, from Tayca,
after treated micronized titanium dioxides with sodium hexametaphosphates, such as, the product “Microtitanium Dioxide MT 150 W” from Tayca.

The treated micronized titanium dioxides employed for the combination may also be after treated with:
octyltrimethoxysilanes; such as, the product Tego Sun T 805 from Evonik Goldschmidt GmbH,
silicon dioxide; such as, for example, the product Parsol T-X from DSM,
aluminium oxide and stearic acid; such as, the product UV-Titan M160 from Sachtleben,
aluminium and glycerin; such as, the product UV-Titan from Sachtleben,
aluminium and silicone oils, such as, the product UV-Titan M262 from Sachtleben,
sodium hexametaphosphate and polyvinylpyrrolidone,
polydimethylsiloxanes, such as, the product 70250 Cardre UF TiO2SI3” from Cardre,
polydimethylhydrogenosiloxanes, such as, for example, the product Microtitanium Dioxide USP Grade Hydrophobic” from Color Techniques.

In a particular embodiment, compositions of the invention may include untreated zinc oxides, such as, the product Z-Cote from BASF (Sunsmart), Nanox from Elementis. In another particular embodiment, compositions of the invention may include after treated zinc oxides, such as, the following products:
“Zinc Oxide CS-5” from Toshibi (ZnO after treated with polymethylhydrogeno-siloxanes);
Nanogard Zinc Oxide FN from Nanophase Technologies;
“SPD-Z1” from Shin-Etsu (ZnO after treated with a silicone-grafted acrylic polymer, dispersed in cyclodimethylsiloxanes;
“Escalol Z100” from ISP (aluminium oxide-after treated ZnO dispersed in an ethylhexyl methoxycinnamate/PVP-hexadecene/methicone copolymer mixture); and
“Fuji ZNO-SMS-10” from Fuji Pigment (ZnO after treated with silicone dioxide and polymethylsilesquioxane).

In one embodiment, compositions of the invention include ZinClear IM 50AB in an amount about 5 wt. % to about 50 wt. %, about 15 wt. % to about 40 wt. %, or about 25 wt. % to about 35 wt. %, based on total weight of the topical composition.

In another particular embodiment, compositions of the invention may include untreated cerium oxide micropigment, for example with the name “Colloidal Cerium Oxide” from Rhone Poulenc. In another particular embodiment, compositions of the invention may include untreated and/or after treated iron oxides with the name Nanogar from Arnaud.

By way of example, it is also possible to employ mixtures of various metal oxides, such as, for example, titanium dioxide and cerium oxide, with and without aftertreatment, such as, for example, the product Sunveil A from Ikeda. In addition, mixtures of aluminium oxide, silicone dioxide and silicone-after treated titanium dioxide, zinc oxide mixtures, such as, the product UV-Titan M261 from Sachtleben, can also be used in combination with the UV protection agents according to the invention.

In certain embodiments, topical compositions described herein may include an active agent selected from sunscreen, an antibiotic, a steroid, hydroquinone, or a combination thereof. Any suitable sunscreen may be utilized, such as any of the sunscreening agents and UV screening agents described herein. In one embodiment, the sunscreen may be an inorganic mineral sunscreen, such as zinc oxide.

In one embodiment, the topical composition may include a steroid, which may be a fluorinated steroid. Suitable steroids may include, without limitations, corticosteroids, alclometasone, dexamethasone, flumethasone, hydrocortisone, hydrocortisone-21-monoesters, hydrocortisone-21-acetate, hydrocortisone-21-butyrate, hydrocortisone-21-propionate, hydrocortisone-21-valerate, hydrocortisone-17,21-di esters, hydrocortisone-17,21-diacetate, hydrocortisone-17-acetate-i-butyrate, hydrocortisone-17,21-dibutyrate, prednisolone, methylprednisolone, betamethasone benzoate, betamethasone diproprionate, clobetasol propionate, diflorasone diacetate, fluocinonide, fluticasone propionate, mometasone furoate, triamcinolone acetonide, topical corticosteroids, hydroxyltriamcinolone, alpha-methyl dexamethasone, dexamethasone-phosphate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, medrysone, amcinafel, amcinafide, betamethasone, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisone, beclomethasone dipropionate, triamcinolone, or any combination thereof.

In one embodiment, the topical composition comprises, consists, or consists essentially of an occlusion forming agent that is hydrophobic (e.g., comprising one or more silicone based components), one or more proteolytic enzymes, and an antioxidant system comprising polyphenol antioxidants (e.g., green tea polyphenols), wherein the topical composition has less than 10 wt. % water, based on total weight of the topical composition. The topical composition may further comprise, consist, or consist essentially of at least one cosmetically acceptable excipient. The topical composition may further comprise, consist, or consist essentially of at least one active agent.

In one embodiment, the topical composition comprises, consists, or consists essentially of an occlusion forming agent that is hydrophobic (e.g., comprising one or more silicone based components), one or more proteolytic enzymes, and an antioxidant system comprising polyphenol antioxidants (e.g., green tea polyphenols), wherein the topical composition has less than 8 wt. % urea, based on total weight of the topical composition. The topical composition may further comprise, consist, or consist essentially of at least one cosmetically acceptable excipient. The topical composition may further comprise, consist, or consist essentially of at least one active agent.

In one embodiment, the topical composition comprises, consists, or consists essentially of an occlusion forming agent that is hydrophobic (e.g., comprising one or more silicone based components), one or more proteolytic enzymes, and an antioxidant system comprising polyphenol antioxidants (e.g., green tea polyphenols), wherein the topical composition is a homogenous suspension. The topical composition may further comprise, consist, or consist essentially of at least one cosmetically acceptable excipient. The topical composition may further comprise, consist, or consist essentially of at least one active agent.

The topical composition may further comprise, consist, or consist essentially of cosmetic wax (C30-45 alkyl methicone), silicone resin, phenyl trimethicone, one or more proteolytic enzymes (e.g., papain, bromelain), antioxidant system (e.g., green tea polyphenols, vitamin E, allantoin), carrier or stabilizer (e.g., isononyl isononanoate, silicone organic elastomer blend Isodecyl Neopentanoate (and) Dimethicone/Bis-Isobutyl PPG-20 Crosspolymer, HDI/trimethylol hexyllactone crosspolymer (and) silica), sunscreen (e.g., zinc oxide), thickener (e.g., glyceryl behenate), and optionally a preservative (e.g., phenoxyethanol).

The topical compositions described herein increase patient compliance due to their aesthetically pleasing texture evidenced by the compositions being washable, dry, non-sticky, non-tacky, non pore-blocking, and hypoallergenic.

### Methods of Use

The topical compositions described herein can be used to treat various scars, including immature scars and mature scars, hypertrophic scars (linear or widespread), and keloids (e.g., minor keloids and major keloids).

The term “immature scars” refers to scar at various earlier stages of formation of scar tissue. Earlier stage of scar tissue formation (proliferation) occurs as blood vessels form and a special cell type, called fibroblasts, generate new fibrous tissue. Collagen is deposited into the region, establishing granulation tissue on which dermal tissue forms covering the area. A delay in this process (epithelialization) can result in more pronounced scarring, whereas good progress through the wound healing cascade helps to minimize the incidence of scar formation. The new tissue resulting from this stage is deposited quickly, lacking alignment; if the tissue remains this disorganized, less-densely packed state, a raised or “hypertrophic” scar could be the result. Immature scars may be red, itchy, and/or painful.

The term “mature scars” refers to a later time point in the scar formation and development process, such as after the scar has undergone a remodeling phase. In the remodeling phase, sometimes called maturation, excess tissue is removed by cellular physiologic processes, including the action of fibroclasts. Unlike the tissue-generating fibroblasts, fibroclasts help to break down and remove superfluous tissue. Acting together, fibroclasts and fibroblasts enable tissue remodeling. Raised scars lower as the collagen and connective tissue fibers align to the appropriate form, becoming more organized and dense. This phase can be influenced by stress and movement, and is a component of wound contraction. Certain mature scars may flatten with time but others may remain problematic.

Hypertrophic scars, characterized by an erythematous (red and inflamed), raised appearance confined to the border of injury, can be classified as linear (e.g., along a surgical incision) or widespread (e.g., burns or other injuries affecting skin expanses). Some scars are classified as keloids, which are characterized by focally raised scars that can expand beyond the immediate area of injury, and often bring pain and itch to the patient. These may occur soon after injury or be delayed, and typically do not abate on their own. Doctors may further classify keloids as minor or major based on the protruding dimension and other features. Minor keloids are only slightly raised and generally have limited spread beyond one year. Major keloids generally protrude more than 5 millimeters, and can continue to expand over the years.

In certain embodiments, the present disclosure is directed to a method of brightening a skin and/or reducing discoloration (e.g., red or purple discoloration) in a scar area of a subject by administering to the skin of a subject an effective amount of any of the topical compositions described herein, wherein the scar is brightened or discoloration is reduced after administration of the topical composition.

In certain embodiment, the present disclosure is directed to a scar treatment regimen retroactive method (after a scar has already formed) that includes administering to a skin of a subject that has been subjected to a dermatological procedure an effective amount of any of the topical compositions described herein.

In certain embodiment, the present disclosure is directed to a scar treatment regimen prophylactic method (to prevent formation of a scar) that includes administering to a skin of a subject that will be subjected to a dermatological procedure an effective amount of any of the topical compositions described herein.

Dermatological procedure that may benefit from prior administration (i.e., pre procedure) or subsequent administration (i.e., post procedure) of the topical compositions described herein include, without limitations, dermatological procedures used for scar remediation, microneedling, dermaplaning, dermabrasion, microdermabrasion, laser treatment (e.g., steroid injections).

In certain embodiments, the methods described herein further include performing the dermatological procedure after administration of the topical composition and/or before administration of the topical composition, depending on the treatment regimen. In certain embodiments, the topical compositions described herein is helpful in improving the outcomes of dermatological procedures, e.g., by speeding healing, quelling discomfort, quenching reactive oxygen species (ROS), inducing collagen formation, or any combination thereof.

As used herein, the terms “treatment” or “treating” with respect to a skin condition generally mean “having positive effect on a skin condition” and encompass reduction, amelioration, and/or alleviation of at least one symptom of a skin condition (e.g., scar), a reduction, amelioration, and/or alleviation in the severity of the skin condition (e.g., scar), or delay, prevention, or inhibition of the progression of the skin condition (e.g., scar), or the perception thereof. Treatment, as used herein, therefore does not require total curing of the condition (e.g., scar). A formulation of the present disclosure that is useful for treatment of a skin condition (e.g., scar), or a method of treating a skin condition (e.g., scar), need only reduce the severity of a skin condition (e.g., scar), reduce the severity of symptoms associated therewith, provide improvement to a patient's quality of life, or delay, prevent, or inhibit the onset of one or more symptoms of a skin condition (e.g., scar). As used herein, these terms also encompass aesthetic improvements to the scar upon application of the disclosed formulations having a combination of, for example, a hydrophobic occlusion forming agent, proteolytic enzyme, and polyphenol antioxidants (e.g., green tea polyphenols).

As used herein, the phrase “effective amount” refers to an amount of a topical composition of the present disclosure, or component thereof, effective to treat a skin condition (e.g., scar) as noted above, including a range of effects, from a detectable local improvement in an area of topical application to substantial relief of symptoms to an improvement in one or more aesthetic criteria, including, but not limited to, a perceived improvement in appearance of scar, scar roughness, scar elasticity, scar firmness, scar volume, scar size, scar color, scar's regenerative and renewal process, redness, dryness, and the like.

The effective amount will vary with the particular scar being treated, the severity of the scar, the duration of the treatment, the specific components of the composition being used, and other factors. In certain embodiments, the topical compositions described herein are suitable for administration by frequent periodic application, such as by a once, twice, thrice or four times daily application or more, e.g., for a duration of at least 1 day, at least 3 days, at least 5 days, at least 7 days, at least 10 days, at least 14 days, at least 21 days, at least 30 days, about 1 day to about 90 days, about 3 days to about 60 days, or about 5 days to about 30 days, any sub-range therein, and so on. Accordingly, in certain embodiments, the methods described herein further include periodically repeating the administration of the topical composition.

In certain embodiments, the skin's flexibility in the scar area and around the scar area is improved after continuous administration of the topical compositions described herein for a duration of about 1 day to about 90 days, about 3 days to about 60 days, or about 5 days to about days, or any sub-range therein.

In certain embodiments, the topical compositions described herein are suitable for a pre or post procedure administration, such as before or after a dermatological procedure (e.g., laser, microneedling, microdermabrasion, dermabrasion, dermaplaning, dermasanding, or a chemical peel), before or after a cosmetic procedure, before or after a surgical procedure, or in combination with a scar revisions procedure.

In certain embodiments, methods described herein can be used to treat, prevent, or minimize the appearance of new and old scars of varying severity, including hypertrophic scar and keloids. Similarly, in certain embodiments, methods described herein can be used to treat, prevent, or minimize the appearance of scars resulting from surgical or cosmetic procedures, trauma, wounds, or burns.

In certain embodiments, methods described herein can be used to help prevent and/or treat sunburn, decrease risk of skin cancer and early skin aging caused by the sun.

### Method of Preparation

The instant disclosure is also directed to a method of preparing any of the topical compositions described herein. The method includes producing any of the topical compositions described herein by combining a hydrophobic occlusion forming agent, a proteolytic enzyme, and polyphenol antioxidants (e.g., green tea polyphenols), along with at least one cosmetically acceptable excipient. The topical composition may be formulated into an ointment serum, gel, lotion, cream, pad applied formulation, and the like.

In certain embodiments, the process includes forming a first mixture that includes an occlusion forming agent, such as a hydrophobic occlusion forming agent having a water solubility of less than 25% (e.g., including one or more silicone based components as described hereinabove). If a sunscreen is included in the topical composition, it may be added to the first mixture. The first mixture may be formed at a temperature of about 50° C. to about 100° C., about 60° C. to about 95° C., or about 75° C. to about 90° C. The first mixture may be mixed and homogenized for a duration sufficient to achieve a uniform mixture.

The process may further include forming a second mixture that includes one or more of the proteolytic enzymes described hereinabove. The second mixture may be formed at a temperature of about 75° C. to about 125° C., from about 80° C. to about 115° C., or from about 90° C. to about 110° C. The second mixture may subsequently be cooled to the temperature of the first mixture so that the two mixtures can be combined (e.g., by adding the second mixture to the first mixture) to form a third mixture.

The process may further include adding polyphenol antioxidants (e.g., green tea polyphenols) and/or other antioxidants in the antioxidant system into the third mixture. Each antioxidant in the antioxidant system may be added individually (i.e., one at a time), and the third mixture may be mixed and homogenized between each addition to achieve a uniform third mixture.

The process may further include adding at least one additional cosmetically acceptable excipient selected from a silicone resin, a stabilizer or carrier, a preservative, or a combination thereof. The silicone resin, stabilizer or carrier, and preservative may be added sequentially, one at a time, with mixing and optionally homogenization between additions, while also gradually cooling the mixture to a temperature of about 25° C. to about 55° C., about 30° C. to about 50° C., or about 35° C. to about 45° C.

The final topical composition may be assessed to confirm that a uniform, dispersed, homogenous suspension with no lumps has been formed.

### ILLUSTRATIVE EXAMPLES

The following examples are set forth to assist in understanding the invention and should not, of course, be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### Example 1—Topical Scar Treatment Composition and Process of Preparation

A topical composition in accordance with embodiments described herein was formulated into a scar treatment topical composition described in Table 1 below. The topical composition was an ointment. Some of the ingredients listed in Table 1 existed in the composition as a two phase suspension.

TABLE 1
Scar Treatment Composition

| | | | | Most |
|---|---|---|---|---|
| | | | Preferred | Preferred |
| | | Amount | Amount | Amount |
| Ingredient | Function | (wt. %*) | (wt. %*) | (wt. %*) |
| Cosmetic Wax | Rheology | 1-30 | 2-15 | 3-5 |
| (e.g., C30-45 alkyl | Modifier and | | | |
| methicone-Dowsil | Occlusion | | | |
| AMS C30) | Forming | | | |
| | Agent | | | |
| Silicone Resin (Dow | Occlusion | 2.5-30 | 5-20 | 7.5-15 |
| Corning TI-7021) | Forming | | | |
| | Agent | | | |
| Phenyl Trimethicone | Occlusion | Above 0- | 2-20 | 5-10 |
| (DC 556) | Forming | 30 | | |
| | Agent | | | |
| Camellia Sinensis | Antioxidant | Above 0- | 0.001-0.5 | 0.005-0.3 |
| (Green Tea) | | 1 | | |
| Polyphenols | | | | |
| Papain | Proteolytic | 0-1 | 0.001-0.5 | 0.005-0.3 |
| | Enzyme | | | |
| Bromelain | Proteolytic | 0-1 | 0.001-0.5 | 0.005-0.3 |
| | Enzyme | | | |
| Glyceryl Behenate | Thickener and | 1-15 | 2.5-10 | 4-7.5 |
| (Comprito l888 | compacting | | | |
| CG ATO) | agent | | | |
| Isononyl | Carrier or | 5-40 | 10-30 | 15-20 |
| isononanoate | | | | |
| (Dermol 99) | Stabilizer | | | |
| A silicone organic | Carrier or | 5-40 | 10-35 | 20-30 |
| elastomer blend | Stabilizer | | | |
| of isodecyl | | | | |
| neopentanoate and | | | | |
| dimethicone/ | | | | |
| bis-isobutyl | | | | |
| PPG-20 cross | | | | |
| polymer | | | | |
| (Dowsil El 8051 IN) | | | | |
| Phenoxyethanol | Preservative | 0-2 | 0-1.5 | 0-0.5 |
| Tocopherol Acetate | Antioxidant | 0-1 | 0.001-0.5 | 0.005-0.2 |
| (Vitamin E) | | | | |
| HDI/Trimethylol | Carrier or | 0-10 | 0.5-5 | 1-3 |
| Hexyllactone | Stabilizer | | | |
| Crosspolymer | | | | |
| (and) Silica | | | | |
| (Kobo BPD-500) | | | | |
| Allantoin | Antioxidant | 0-1 | 0.001-0.5 | 0.005-0.2 |
| Zinc Oxide | Sunscreen | 5-50 | 15-40 | 25-35 |
| (ZinClear IM 50AB) | | | | |

*wt. % are based on the total weight of the composition

The scar treatment ointment composition from Table 1 was prepared by forming a first mixture of: a majority portion of the isononyl isononanoate carrier, glyceryl behenate thickener, cosmetic wax occlusion forming agent, phenyl trimethicone occlusion forming agent, and the sunscreen. The first mixture was mixed until uniform, optionally with a homogenizer, at a temperature of about 75° C. to about 90° C.

Separately, a second mixture was formed by mixing the remainder of the isononyl isononanoate carrier and one or more proteolytic enzymes. The second mixture was mixed until uniform at about 90° C. to about 110° C. Thereafter, the second mixture was cooled to the temperature of the first mixture followed by addition of the second mixture into the first mixture.

### Example 2—Application of Scar Treatment Ointment

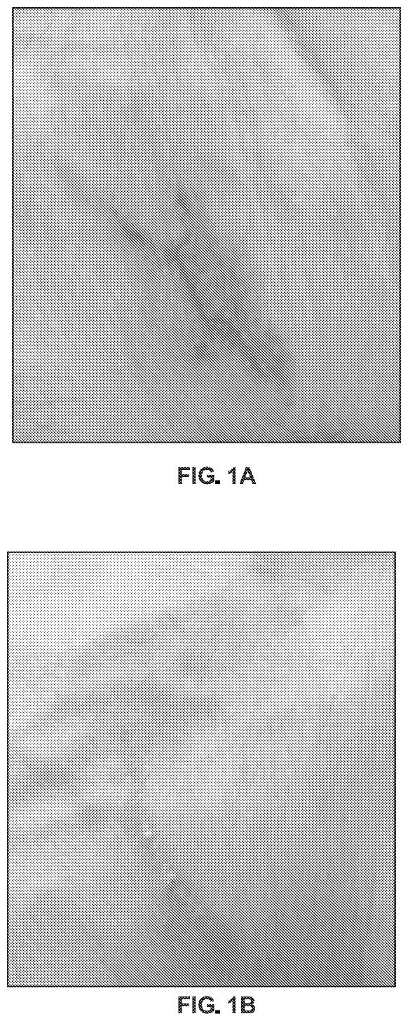

The scar treatment ointment of Table 1 was applied to a wound three times daily for two days. The initial wound is presented in FIG. 1A and the wound after being treated for 48 hours is presented in FIG. 1B with the scar treatment ointment of Table 1. As can be seen in FIGS. 1A and 1B, the wound almost completely healed after being treated with the scar treatment ointment.

### Example 3—Study to Increase Wound Healing and Minimize Scarring

A study was conducted using the hydrophilic scar treatment composition described in Table 2 along with the scar treatment ointment from Table 1. The study was designed to increase wound healing resulting in minimizing the window of potential receiving a scar as well as reducing the appearance of atrophic scars.

TABLE 2
Hydrophilic Scar Treatment Composition

| | | | | Most |
|---|---|---|---|---|
| | | | Preferred | Preferred |
| | | Amount | Amount | Amount |
| Ingredient | Function | (wt. %*) | (wt. %*) | (wt. %*) |
| Glycerin | Hydrophilic | 70-99 | 90-98 | 95-97 |
| | Occlusion | | | |
| | Forming | | | |
| | Agent | | | |
| Sensogel 200 | Binding | 0.1-10 | 1-5 | 2-3 |
| (HEA-sodium | Agent | | | |
| acryloyldimethyltaurate- | | | | |
| steareth-20 methacrylate | | | | |
| copolymer, C13-14 | | | | |
| isoparaffin, polysorbate | | | | |
| 80, decyl glucoside, | | | | |
| and glycerin) | | | | |
| Camellia Sinensis | Antioxidant | Above 0- | 0.001-0.5 | 0.005-0.3 |
| (Green Tea) | | | | |
| Polyphenols | | 1 | | |
| Papain | Proteolytic | 0-1 | 0.001-0.5 | 0.005-0.3 |
| | Enzyme | | | |
| Bromelain | Proteolytic | 0-1 | 0.001-0.5 | 0.005-0.3 |
| | Enzyme | | | |
| Phenoxyethanol | Preservative | 0-2 | 0-1.5 | 0-0.5 |
| Tocopherol Acetate | Antioxidant | 0-1 | 0.001-0.5 | 0.005-0.2 |
| (Vitamin E) | | | | |
| Allantoin | Antioxidant | 0-1 | 0.001-0.5 | 0.005-0.2 |
| DC 200 Fluid 350 cst | Silicone | 0-10 | 0-5 | 0-1 |
| (Dimethicone) | component | | | |

*wt. % are based on the total weight of the composition

The hydrophilic scar treatment composition was applied immediately post incidence, abrasion or incision and was then applied 3 times daily over the course of 14 days. The hydrophilic scar treatment composition was applied directly to the wound or incision, below Steri-strip tape or above micropore tape, while the scar treatment ointment was applied directly to the wound or incision. After 14 days, then scar treatment ointment of Table 1 was applied 2-3 times a day for the remainder of the study.

Clinical grading of efficacy parameters at baseline and weeks 2, 4, 8 and 12 were obtained. Further, before and after photos were taken at 2, 4, 6, 8 and 12 weeks. The subjects also completed a self-assessment questionnaire at weeks 2, 4, 8 and 12. The clinical grading of objective irritation parameters and subject reporting of subjective irritation parameters at baseline and weeks 2, 4, 8 and 12 were obtained. The study also monitored any adverse events throughout the course of the study.

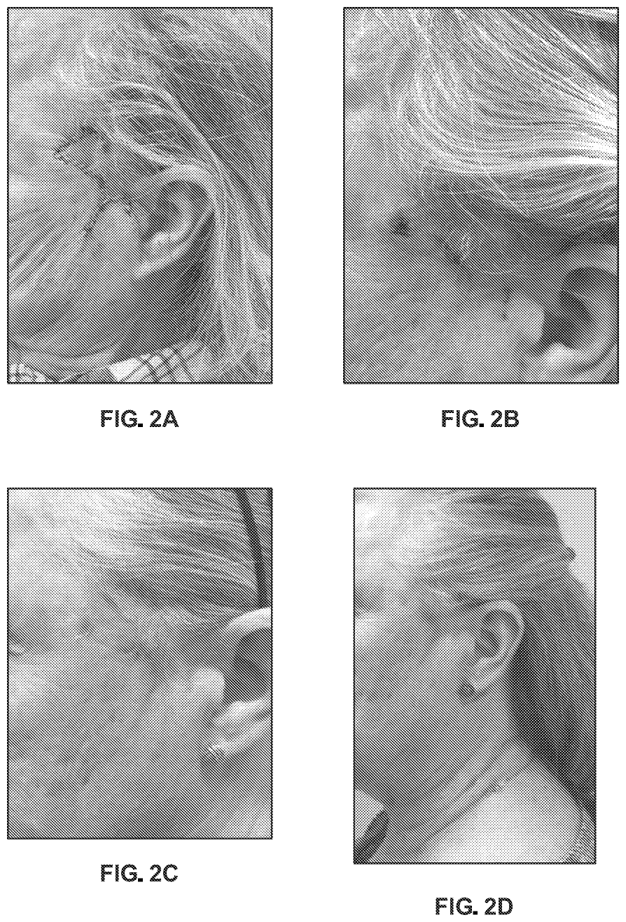

In one subject, the hydrophilic scar treatment composition was applied 3 times daily over the course of 14 days, after a MOHS surgery. After 14 days, i.e. 2 weeks, the scar treatment ointment was applied 2-3 times a day for an additional 10 weeks, for a total treatment of 12 weeks. As can be seen in FIGS. 2A-2D, photos were taken of one subject at baseline, 2 week, 6 week and 12 week point, respectively. Over the course of the 12 week treatment with both the hydrophilic scar treatment composition and scar treatment ointment, the scar was almost completely healed.

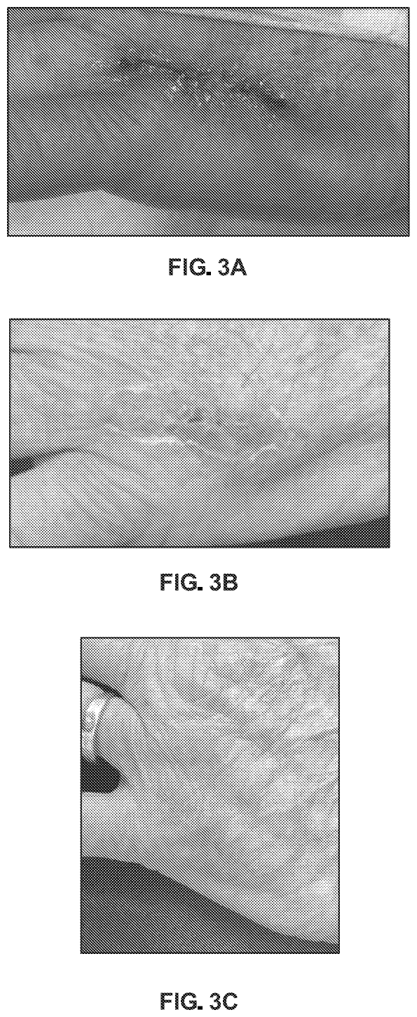

In another subject, the hydrophilic scar treatment composition was applied 3 times daily over the course of 14 days, after a MOHS surgery on the hand. As can be seen in FIGS. 3A and 3B, the wound healed at least about 80% over the course of 14 days. Also, no scarring formed in the healed areas. After the 14 days, the scar treatment ointment was applied for another 10 weeks. As can be seen in FIG. 3C, the wound healed completely with minimal scarring after 12 weeks.

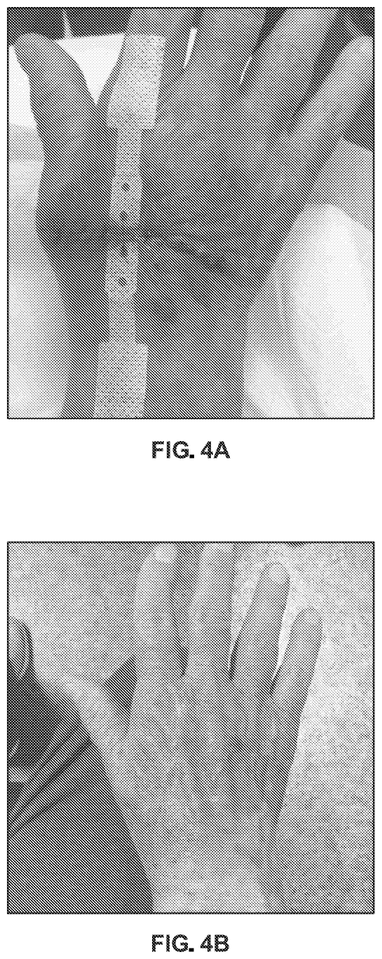

In another subject who had a MOHS surgery, the hydrophilic scar treatment composition was applied 3 times daily over the course of 14 days. After the 14 days, the scar treatment ointment was applied for another 10 weeks. A baseline photo of this subject can be seen in FIG. 4A, while FIG. 4B represents a photo after 12 weeks of treatment according to the study. As can be seen in FIGS. 4A and 4B, the wound completely healed after 12 weeks with minimal scarring.

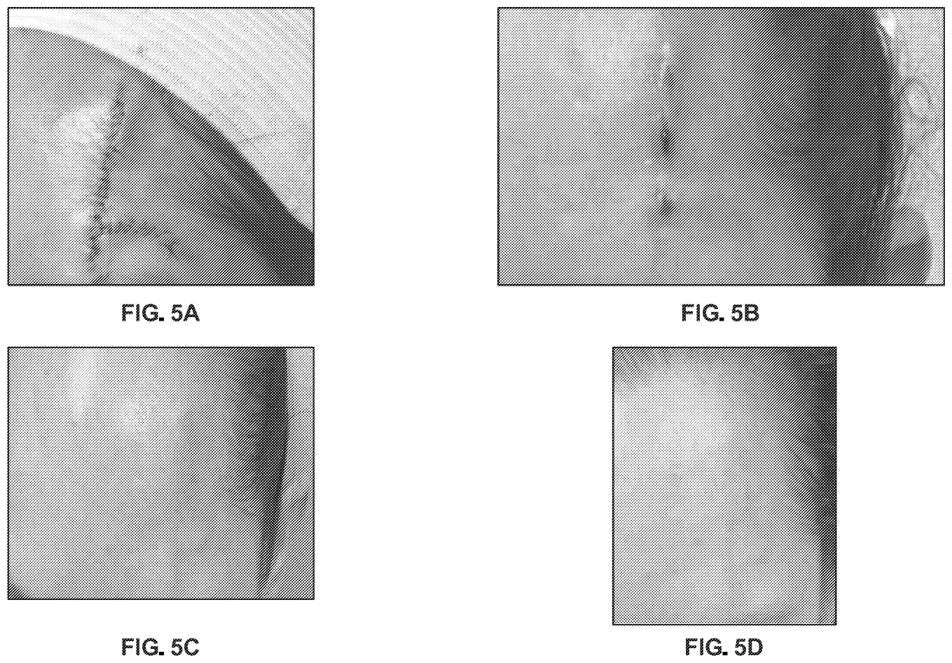

In another subject, who had a MOHS surgery on the forehead, the hydrophilic scar treatment composition was applied 3 times daily over the course of 14 days. After the 14 days, the scar treatment ointment was applied for another 10 weeks. A baseline photo of this subject can be seen in FIG. 5A. FIG. 5B represents a photo after 2 weeks of treatment with the hydrophilic scar treatment composition. In FIG. 5C, a photo of the forehead was taken after 8 weeks total treatment, i.e. 6 weeks after the 2 week photo in FIG. 5B. In FIG. 5D, a photo of the forehead was taken after 12 weeks of treatment according to the study. As can be seen in FIGS. the wound was completely healed after 12 weeks with minimal scarring.

Thereafter, one or more antioxidants forming the antioxidant system that includes green tea polyphenols was added one at a time into the combined first and second mixture, with homogenization or mixing between additions, until uniform.

Subsequently, a silicone resin occlusion forming agent was added, and the composition was mixed and homogenized until uniform and well dispersed.

Subsequently, the composition was cooled to a temperature of about 60° C. to about ° C., and a silicone organic elastomer blend of isodecyl neopentanoate and dimethicone/bis-isobutyl PPG-20 cross polymer carrier or stabilizer was added, the composition was mixed and homogenized until uniform and well dispersed.

Subsequently, the composition was cooled to a temperature of about 40° C. to about ° C., and a silica carrier or stabilizer along with a preservative (if included) was added, the composition was cooled while being mixed and homogenized at a temperature of about 30° C. to about 50° C.

For simplicity of explanation, the embodiments of the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

Reference throughout this specification to numerical ranges should not be construed as limiting and should be understood as encompassing the outer limits of the range as well as each number and/or narrower range within the enumerated numerical range.

The term “about”, when referring to a physical quantity, is to be understood to include measurement errors within, and inclusive of 10%. For example, “about 100° C.” should be understood to mean “100±10° C.”.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. A topical composition comprising
an occlusion forming agent comprising one or more silicone based components;
one or more proteolytic enzymes; and
an antioxidant system comprising green tea polyphenols;
wherein the topical composition has less than 10 wt. % water, based on total weight of the topical composition.

2. The topical composition of claim 1, wherein the one or more proteolytic enzymes are selected from papain, bromelain, or a combination thereof.

3. The topical composition of claim 1, wherein the one or more proteolytic enzymes is present in the topical composition in an effective amount for scavenging over production of scar forming proteins and tissues.

4. The topical composition of claim 1, wherein the one or more proteolytic enzymes is present in the topical composition in an amount of from above 0 wt. % and up to about 1 wt. %, based on total weight of the topical composition.

5. The topical composition of claim 1, wherein the green tea polyphenols comprise one or more of epigallocatechin gallate, epigallocatechin, epicatechin gallate, catechin, epicatechin, cis and trans isomers thereof, salts thereof, equivalent derivatives thereof, and a combination thereof.

6. (canceled)

7. The topical composition of claim 1, wherein the antioxidant system is present in the topical composition in an amount of above 0 wt. % and up to about 1 wt. %, based on total weight of the topical composition.

8. The topical composition of claim 7, wherein the antioxidant system further comprises additional antioxidants selected from the group of cinnamic acid, ferulic acid, caffeic acid, p-coumaric acid, sinapinic acid, gallic acid, delphinidin, luteolin, quercetin, cyaniding, taxifolin, kaempferol, malvidin, hesperidin, pelargonidin, apigenin, naringenin, chrysin, ergothioneine, glutathione, emblica, tocopherol, allantoin, cis and trans isomers thereof, salts thereof, equivalent derivatives thereof, and combinations thereof.

9. The topical composition of claim 8, wherein the antioxidant system further comprises tocopherol, allantoin, or a combination thereof.

10. (canceled)

11. The topical composition of claim 1, further comprising an occlusion forming agent.

12.-20. (canceled)

21. The topical composition of claim 1, further comprising a stabilizer or a carrier.

22. The topical composition of claim 21, wherein the stabilizer or carrier comprises isononyl isononanoate, a silicone organic elastomer blend of isodecyl neopentanoate and dimethicone/bis-isobutyl PPG-20 cross polymer, or a combination thereof.

23. (canceled)

24. (canceled)

25. The topical composition of claim 1, further comprising an active agent selected from a sunscreen, an antibiotic, a steroid, hydroquinone, or a combination thereof.

26.-28. (canceled)

29. The topical composition of claim 1, wherein the topical composition is hypoallergenic.

30. (canceled)

31. The topical composition of claim 1, wherein the topical composition has less than about 8 wt. % water, based on total weight of the topical composition.

32. The topical composition of claim 1, wherein the topical composition has less than about 8 wt. % urea, based on total weight of the topical composition.

33. The topical composition of claim 1, wherein the topical composition is a homogenous suspension.

34. (canceled)

35. A topical composition comprising
an occlusion forming agent comprising one or more silicone based components;
one or more proteolytic enzymes; and
an antioxidant system comprising green tea polyphenols;
wherein the topical composition is a homogenous suspension.

36. A method for treating, preventing, or minimizing the appearance of a scar comprising administering to a patient in need thereof the topical compositions of claim 1.

37.-42. (canceled)

43. A process for preparing the topical compositions of claim 1, comprising:
forming a first mixture comprising the occlusion forming agent comprising one or more silicone based components;
forming a second mixture comprising the one or more proteolytic enzymes;
combining the first mixture and the second mixture to form a third mixture; and
adding the antioxidant system comprising the green tea polyphenols to the third mixture.

44.-50. (canceled)

51. A topical composition comprising
A hydrophobic occlusion forming agent having less than about 25% water solubility;
one or more proteolytic enzymes; and
an antioxidant system comprising green tea polyphenols;
wherein the topical composition has less than 10 wt. % water, based on total weight of the topical composition.

52.-56. (canceled)
